(19) United States
(12) Patent Application Publication
(10) Pub. No.: US 2005/0171194 A1
(43) Pub. Date: Aug. 4, 2005

(54) Enlargement of mucocutaneous or cutaneous organs and sites with topical compositions

(76) Inventors: Ruey J. Yu, Chalfont, PA (US); Eugene J. Van Scott, Abington, PA (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 11/006,822

(22) Filed: Dec. 8, 2004

Related U.S. Application Data
(60) Provisional application No. 60/527,307, filed on Dec. 8, 2003.
(60) Provisional application No. 60/570,895, filed on May 14, 2004.

(51) Int. Cl. A61K 31/366; A61K 31/19

(52) U.S. Cl. 514/460; 514/473; 514/557

(57) ABSTRACT

Compositions comprising a hydroxycarboxylic acid, N-acyl-aldosamine, N-acylamino acid or related compound on topical application are beneficial to plump and pout lips, enhance and firm eyelids, enlarge and augment breasts, elongate and expand penis. Because of antioxidant property, certain hydroxycarboxylic acids, N-acyl-aldosamines, N-acylamino acids and related compounds also are useful for topical administration to prevent occurrence of breast cancer or other forms of tumors and cancers.

This application claims priority under 35 U.S.C. §119 to Provisional Patent Application No. 60/527,307, filed on Dec. 8, 2003, and Provisional Patent Application No. 60/570,895, filed on May 14, 2004, the disclosures of each of which are incorporated by reference herein in their entireties.

### FIELD OF THE INVENTION

Embodiments described herein relate to the use of compositions comprising a hydroxycarboxylic acid, N-acyl-aldosamine, N-acylamino acid, and/or compounds related thereto, for topical administration to a mammal. Topical administration is believed to plump, enhance, enlarge and/or elongate the mucous membrane or skin organs and sites which include lips, eyelids, breasts and penis.

### DESCRIPTION OF RELATED ART

U.S. Pat. No. 5,091,171 entitled “Amphoteric Compositions and Polymeric Forms of Alpha Hydroxyacids, and Their Therapeutic Use” describes preventive as well as therapeutic treatments to alleviate cosmetic conditions and symptoms of dermatologic disorders with amphoteric compositions containing alpha hydroxyacids, alpha ketoacids related compounds or polymeric forms of hydroxyacids. U.S. Pat. No. 5,547,988 entitled “Alleviating Signs of Dermatological Aging with Glycolic Acid, Lactic Acid or Citric Acid” describes the use of alpha-hydroxyacids to alleviate or improve signs of skin, nail and hair changes associated with intrinsic or extrinsic aging. U.S. Pat. No. 5,385,938 entitled “Method of Using Glycolic Acid for Treating Wrinkles” describes the use of glycolic acid for topical treatment of wrinkles. U.S. Pat. No. 5,258,391, entitled “Phenyl Alpha Acyloxyalkanoic Acids, Derivatives and Their Therapeutic Use” describes the use of topical compositions containing phenyl alpha acyloxyalkanoic acids and derivatives to enhance the keratinization of nails, skin, lips and other mucous membranes.

U.S. Pat. No. 5,665,776 entitled “Additives Enhancing Topical Actions of Therapeutic Agents” describes the use of hydroxycarboxylic acids to enhance the therapeutic effects of cosmetic or pharmaceutical agents. U.S. Pat. No. 5,889,054 entitled “Method of Using Beta Hydroxyacids for Treating Wrinkles” describes the use of compositions comprising a beta-hydroxyacid for topical treatment of skin changes associated with aging. U.S. Pat. No. 6,060,512 entitled “Method of Using Hydroxycarboxylic Acids or Related Compounds for Treating Skin Changes Associated with Intrinsic and Extrinsic Aging” describes the use of compositions comprising a hydroxycarboxylic acid for topical treatment of skin changes associated with intrinsic and extrinsic aging. U.S. Pat. Nos. 6,335,023 B1 and 6,740,327 B2 entitled “Oligosaccharide Aldonic Acids and Their Topical Use” describes and claims the use of compositions comprising an oligosaccharide aldonic acid for topical treatment of cosmetic conditions and dermatological disorders. U.S. Pat. No. 6,767,924 entitled “Method of Using Hydroxycarboxylic Acids or Related Compounds for Treating Skin Changes Associated with Intrinsic and Extrinsic Aging” describes and claims the use of compositions comprising a polyhydroxy acid in an amphoteric system for topical treatment of skin changes associated with intrinsic and extrinsic aging.

U.S. Pat. No. 5,641,475 entitled “Antiodor, Antimicrobial and Preservative Compositions and Methods of Using Same” describes and claims the use of topical compositions containing a bioactive cosmetic, dermatologic or preservative agent and aryl 2-acetoxyethanoic acid effective as a synergist or amplifier of the agent. U.S. Pat. No. 5,643,949 entitled “Phenyl Alpha Acyloxyalkanoic Acids, Derivatives and Their Therapeutic Use” describes and claims the use of topical compositions containing a cosmetic or dermatologic drug for topical administration to nails, skin and lips and an amount of a phenyl alpha acyloxyalkanoic acid or derivatives effective to enhance the cosmetic or therapeutic effect of the dermatologic drug.

PCT Application No. PCT/US96/16534, filed Oct. 16, 1996, entitled “Topical Compositions Containing N-Acetylcysteine and Odor Masking Materials,” describes topical compositions comprising from 0.01% to 50% of N-acetylcysteine or a derivative of N-acetylcysteine, from 0.01% to 0.5% of an odor masking material, and a topical carrier to improve the appearance of skin. U.S. Pat. No. 6,159,485 entitled “N-Acetyl Aldosamines, N-Acetylamino Acids and Related N-Acetyl Compounds and Their Topical Use” and U.S. Pat. No. 6,524,593 B1 entitled “N-Acetyl Aldosamines and Related N-Acetyl Compounds, and Their Topical Use,” describes and claims the use of compositions comprising N-acetylamino acids and N-acetyl aldosamines for topical treatment of cosmetic conditions and dermatological disorders. The disclosures of each of the aforementioned United States patents are incorporated by reference herein in their entireties.

The description herein of disadvantages and problems associated with known compositions and methods is not intended to limit the scope of the invention. Indeed, certain embodiments described and claimed herein may include one or more known compositions or methods without suffering from these disadvantages and/or problems.

### SUMMARY

The present inventors have discovered that hydroxycarboxylic acids, N-acyl-aldosamines, N-acylamino acids and related compounds have much broader utilities than being described in the past. In accordance with a feature of an embodiment of the invention, there is provided a composition comprising a hydroxycarboxylic acid, N-acyl-aldosamine, N-acylamino acid or related compounds that is useful for topical application to a desired area, to provide the following advantageous effects: plump and pout lips; enhance and firm eyelids; enlarge and augment breasts; and elongate and expand the penis.

Embodiments of the invention also include a method of plumping and pouting lips, a method of enhancing and firming eyelids, a method of enlarging and augmenting breasts, and a method of elongating and expanding the penis comprising administering the affected area a composition comprising at least one selected from the group consisting of a hydroxycarboxylic acid, N-acyl-aldosamine, N-acylamino acid or related compounds, in an effective amount and for an effective period of time to achieve the desired effect.

These and other features of various embodiments of the invention will become readily apparent to those skilled in the art upon review of the detailed description that follows.

### DESCRIPTION OF THE EMBODIMENTS

The inventors have discovered that hydroxycarboxylic acids, N-acyl-aldosamines, N-acylamino acids and related compounds on topical application to the affected area are useful to plump and pout lips, enhance and firm eyelids, enlarge and augment breasts, and elongate and expand the penis. The inventors also have discovered that certain hydroxycarboxylic acids, N-acyl-aldosamines, N-acylamino acids and related compounds are antioxidant substances, and they are beneficial for topical administration to prevent or treat breast cancer and other forms of tumors and cancers caused by free radicals, radiations, oxidation, photodamage and carcinogenesis. The organic compounds of the present invention, which are useful and beneficial for topical treatment to enlarge, plump or elongate mucocutaneous organs and sites can be classified into three groups: (A) hydroxycarboxylic acids; (B) N-acyl-aldosamines; and (C) N-acylamino acids and related compounds.

Preferred hydroxycarboxylic acids useful in embodiments include alpha-hydroxyacids, beta-hydroxyacids, polyhydroxy acids and aldobionic acids as free acid, salt, amide, ester or lactone form. Representative hydroxycarboxylic acids include glycolic acid, lactic acid, citric acid, beta-hydroxypropanoic acid, beta-hydroxybutanoic acid, tropic acid, ribonolactone, gluconolactone, lactobionic acid and maltobionic acid. Preferred N-acylamino compounds useful in embodiments include N-acyl-aldosamines and N-acylamino acids which include N-acetyl-aldosamines, N-propanoyl-aldosamines, N-acetylamino acids, N-propanoylamino acids as free acid, salt or partial salt with organic or inorganic alkali, amide, ester or lactone form. Preferred N-acyl-aldosamines, N-acylamino acids and related compounds include N-acetyl-glucosamine, N-propanoyl-glucosamine, N-acetyl-cysteine, N-acetyl-cysteine ethyl ester, N-acetyl-glutamine, N-acetyl-arginine, N-acetyl-proline, N-acetyl-prolinamide, N-acetyl-proline esters, N-propanoyl-glutamine, N-propanoyl-proline, N-propanoyl-prolinamide, N-propanoyl-proline ethyl ester; N,S-diacetyl-cysteine; N,S-diacetyl-cysteine methyl ester and N,S-diacetyl-cysteine ethyl ester.

The three groups of compounds mentioned above are further exemplified below.

(A) Hydroxycarboxylic Acids

The hydroxycarboxylic acids useful in embodiments typically are organic hydroxyacids wherein both hydroxyl and carboxyl groups are attached to aliphatic or alicyclic hydrocarbons. In accordance with embodiments, the hydroxyacids and derivatives can be divided into the following groups.
(1) Alpha-hydroxyacids (AHAs)

AHAs are organic carboxylic acids having one hydroxyl group attached directly to the alpha position of aliphatic or alicyclic carbon atom, but not to a benzene or other aromatic ring. On a broader scope, AHAs may include those which have additional carboxyl groups. The AHAs can be divided into three subgroups (a) alkyl AHAs, (b) aralkyl AHAs and (c) polycarboxyl AHAs.
(a) Alkyl AHAs

The side chain radicals attached to the alpha carbon are hydrogen atoms or simple hydrocarbons called alkyl groups. The generic structure can be represented as follows:
R₁R₂C(OH)COOH
where R₁ and R₂ may be independently H or alkyl group. The alkyl AHAs can exist as free acid, salt or partial salt with organic or inorganic alkali, amide, ester, lactone, stereoisomers as D, L and DL or R, S and RS forms when R₁ and R₂ are not identical. The alkyl groups are non-aromatic radicals such as methyl, ethyl, propyl, isopropyl, butyl, pentyl, octyl, lauryl and stearyl.

Representative alkyl AHAs are be listed as follows:
2-hydroxyethanoic acid (glycolic acid);
2-hydroxypropanoic acid (lactic acid);
2-methyl-2-hydroxypropanoic acid (methyllactic acid);
2-hydroxybutanoic acid;
2-hydroxypentanoic acid;
2-hydroxyhexanoic acid;
2-hydroxyheptanoic acid;
2-hydroxyoctanoic acid;
2-hydroxyeicosanoic acid (alpha hydroxyarachidonic acid);
2-hydroxytetraeicosanoic acid (cerebronic acid); and
2-hydroxytetraeicosenoic acid (alpha hydroxynervonic acid).
   (b) Aralkyl AHAs

Aralkyl is an abbreviation of aryl plus alkyl. Aralkyl AHA is formed when a phenyl group or other aromatic ring is attached to the alpha or beta carbon of the alkyl AHA. The generic structure is shown as follows.
R₁R₂C(OH)COOH
where R₁ and R₂ may be independently H, aryl or aralkyl group. The aralkyl AHAs can exist as free acid, salt or partial salt with organic or inorganic alkali, amide, ester, lactone, stereoisomers as D, L and DL or R, S and RS forms when R₁ and R₂ are not identical. The aryl group preferably consists of at least one aromatic radical such as phenyl, diphenyl, biphenyl and naphthyl. The aralkyl group preferably consists of at least one aromatic radical and one non-aromatic radical, such as phenylmethyl (benzyl), phenylethyl, phenylpropyl, diphenylmethyl, diphenylethyl, biphenylmethyl and naphthylmethyl. In any case, the hydroxyl group is attached to the non-aromatic alpha carbon atom.

Representative aralkyl AHAs are as follows:
2-henyl-2-hydroxyethanoic acid (mandelic acid);
2,2-diphenyl-2-hydroxyethanoic acid (benzilic acid);
3-phenyl 2-hydroxypropanoic acid (3-phenyllactic acid); and
2-phenyl-2-methyl-2-hydroxyethanoic acid (atrolactic acid, 2-phenyllactic acid).
   (c) Polycarboxy AHAs

In a polycarboxy alpha hydroxyacid, the AHA may include more than one carboxyl and hydroxyl groups. The generic structure can be shown as follows.
R₁R₂C(OH)COOH
where R₁ and R₂ may be independently H, COOH, CH₂COOH or CHOHCOOH. Suitable polycarboxy AHAs may exist as a free acid, salt or partial salt with organic or inorganic alkali, amide, ester, lactone, stereoisomers as D, L and DL or R, S and RS forms when R₁ and R₂ are not identical. Commonly known polycarboxy AHAs include, but are not limited to:
2-hydroxypropane-1,3-dioic acid (tartronic acid);
2-hydroxybutane-1,4-dioic acid (malic acid);
2,3-dihydroxybutane-1,4-dioic acid (tartaric acid);
2-hydroxy-2-carboxypentane-1,5-dioic acid (citric acid); and isocitric acid.
   (2) Beta-hydroxyacids (BHAS)

BHAs are organic carboxylic acids having one hydroxyl group attached to the beta position of aliphatic carbon atom. The generic structure can be represented as follows:
R₁R₂C(OH)CHR₃COOH
where R₁, R₂, R₃ may be H, alkyl, aryl or aralkyl group. The BHA can exist as free acid, salt or partial salt with organic or inorganic alkali, amide, ester, lactone, stereoisomers as D, L and DL or R, S and RS forms when R₁ and R₂ are not identical or R₃ is not H.

Representative BHAs are listed as follows:
3-hydroxypropanoic acid (β-hydroxypropanoic acid);
3-hydroxybutanoic acid (β-hydroxybutanoic acid);
3-hydroxypentanoic acid; and
3-hydroxy-2-phenylpropanoic acid (tropic acid).
   (3) Polyhydroxy Acids (PHAS)

PHAs preferably are organic carboxylic acids having multiple hydroxyl groups in addition to the alpha-hydroxyl group and commonly exist in the lactone form, such as gluconolactone from gluconic acid. Many PHAs are derived from carbohydrates and are important carbohydrate intermediates and metabolites. PHAs may be divided into three groups (a) aldonic acid, (b) aldaric acid and (c) alduronic acid.
(a) Aldonic Acid

When a common carbohydrate such as glucose, also called aldose, is oxidized at the carbon one position from aldehyde to a carboxyl group, the product is called aldonic acid, or more specifically gluconic acid. The aldonic acid usually has multiple hydroxyl groups. A generic structure can be shown as follows.
R(CHOH)ₙCHOHCOOH
where R is usually H or alkyl group; n an integer from 1-6. The aldonic acids can exist as free acid, salt or partial salt with organic or inorganic alkali, amide, ester, lactone, stereoisomers as D, L and DL or R, S and RS forms. Many aldonic acids form intramolecular lactones by removing one mole of water between the carboxyl group and one hydroxyl group.

The following are representative aldonic acids:
2,3-dihydroxypropanoic acid (glyceric acid);
2,3,4-trihydroxybutanoic acids (stereoisomers; erythronic acid and erythronolactone, threonic acid and threonolactone);
2,3,4,5-tetrahydroxypentanoic acids (stereoisomers; ribonic acid and ribonolactone, arabinoic acid and arabinolactone, xylonic acid and xylonolactone, lyxonic acid and lyxonolactone);
2,3,4,5,6-pentahydroxyhexanoic acids (stereoisomers; allonic acid and allonolactone, altronic acid and altronolactone, gluconic acid and gluconolactone, mannoic acid and mannolactone, gulonic acid and gulonolactone, idonic acid and idonolactone, galactonic acid and galactonolactone, talonic acid and talonolactone); and
2,3,4,5,6,7-hexahydroxyheptanoic acids (stereoisomers; alloheptonic acid and alloheptonolactone, altroheptonic acid and altroheptonolactone, glucoheptonic acid and glucoheptonolactone, mannoheptonic acid and mannoheptonolactone, guloheptonic acid and guloheptonolactone, idoheptonic acid and idoheptonolactone, galactoheptonic acid and galactoheptonolactone, taloheptonic acid and taloheptonolactone).
   (b) Aldaric acid

The aldaric acid has multiple hydroxyl groups attached to the carbon chain surrounded by two carboxyl groups. Many aldaric acids exist as lactones, such as glucarolactone. A generic structure can be shown as follows:
HOOC(CHOH)ₙCHOHCOOH
where n is an integer from 1-4. The aldaric acids can exist as free acid, salt or partial salt with organic or inorganic alkali, amide, ester, lactone, stereoisomers as D, L and DL or R, S and RS forms. Many aldaric acids form intramolecular lactones by removing one mole of water between one carboxyl group and one hydroxyl group.

The following are representative aldaric acids:
2,3-dihydroxybutane-1,4-dioic acids (stereoisomers; erythraric acid and threaric acid, also known as tartaric acid);
2,3,4-trihydroxypentane-1,5-dioic acids (stereoisomers; ribaric acid and ribarolactone, arabaric acid and arabarolactone, xylaric acid and xylarolactone, lyxaric acid and lyxarolactone);
2,3,4,5-tetrahydroxyhexane-1,6-dioic acids (stereoisomers; allaric acid and allarolactone, altraric acid and altrarolactone, glucaric acid and glucarolactone, mannaric acid and mannarolactone, gularic acid and gularolactone, idaric acid and idarolactone, galactaric acid and galactarolactone, talaric acid and talarolactone);
2,3,4,5,6-pentahydroxyheptane-1,7-dioic acids (stereoisomers; alloheptaric acid and alloheptarolactone, altroheptaric acid and altroheptarolactone, glucoheptaric acid and glucoheptarolactone, mannoheptaric acid and mannoheptarolactone, guloheptaric acid and guloheptarolactone, idoheptaric acid and idoheptarolactone, galactoheptaric acid and galactoheptarolactone, taloheptaric acid and taloheptarolactone).
   (c) Alduronic acid

Alduronic acid preferably is obtained from a carbohydrate, e.g., aldose, by oxidation of the terminal carbon to carboxyl group, and the carbon one position remains as an aldehyde group, such as glucuronic acid from glucose. Similar to aldonic acid and aldaric acid, alduronic acids also have multiple hydroxyl groups attached to the carbon chain between two functional groups—one aldehyde and one carboxyl groups in this case. Many alduronic acids exist as lactones, such as glucuronolactone from glucuronic acid. The generic structure can be shown as follows:
HOOC(CHOH)ₙCHOHCHO
where n is an integer from 1-4. The alduronic acids can exist as a free acid, salt or partial salt with organic or inorganic alkali, amide, ester, lactone, stereoisomers as D, L and DL or R, S and RS forms. Many alduronic acids can form intramolecular lactones by removing one mole of water between the carboxyl group and one hydroxyl group.

The following are representative alduronic acids:
erythruronic acid and threuronic acid;
riburonic acid and riburonolactone;
araburonic acid and araburonolactone;
xyluronic acid and xyluronolactone;
lyxuronic acid and lyxuronolactone;
alluronic acid and alluronolactone;
altruronic acid and altruronolactone;
glucuronic acid and glucuronolactone;
mannuronic acid and mannuronolactone;
guluronic acid and guluronolactone;
iduronic acid and iduronolactone;
galacturonic acid and galacturonolactone;
taluronic acid and taluronolactone;
allohepturonic acid and allohepturonolactone;
altrohepturonic acid and altrohepturonolactone;
glucohepturonic acid and glucohepturonolactone;
mannohepturonic acid and mannohepturonolactone;
gulohepturonic acid and gulohepturonolactone;
idohepturonic acid and idohepturonolactone;
galactohepturonic acid and galactohepturonolactone; and
talohepturonic acid and talohepturonolactone.
   (4) Aldobionic Acids (ABAs)

ABAs also are known as bionic acids, and typically include one monosaccharide chemically linked through an ether bond to an aldonic acid. The ABA also may be described as an oxidized form of a disaccharide or dimeric carbohydrate, such as lactobionic acid from lactose. In most ABAs, the carbon at position one of the monosaccharide is chemically linked to a hydroxyl group at different position of the aldonic acid. Therefore, different ABAs or stereoisomers can be formed from two identical monosaccharide and aldonic acid. Similar to PHAs, ABAs have multiple hydroxyl groups attached to carbon chains. ABAs can be represented by the following generic formula:
H(CHOH)ₘ(CHOR)(CHOH)ₙCOOH
where m and n are integers independently from 0-7; R is a monosaccharide. ABAs can exist as free acid, salt or partial salt with organic or inorganic alkali, amide, ester, lactone, stereoisomers as D, L and DL or R, S and RS forms, and can form intramolecular lactones by removing one mole of water between the carboxyl group and one hydroxyl group. The chemical structures of most ABAs are more complicated than the above generic formula.

The ABAs therefore will be represented by their chemical names where appropriate in this descrption: lactobionic acid and lactobionolactone from lactose, isolactobionic acid and isolactobionolactone from isolactose, maltobionic acid and maltobionolactone from maltose, isomaltobionic acid and isomaltobionolactone from isomaltose, cellobionic acid and cellobionolactone from cellobiose, gentiobionic acid and gentiobionolactone from gentiobiose, kojibionic acid and kojibionolactone from kojibiose, laminaribionic acid and laminaribionolactone from laminaribiose, melibionic acid and melibionolactone from melibiose, nigerobionic acid and nigerobionolactone from nigerose, rutinobionic acid and rutinobionolactone from rutinose, sophorobionic acid and sophorobionolactone from sophorose.

The hydroxyacid derivatives useful in embodiments of the invention include ester forms or O-acetyl forms of the hydroxyacids described above. Thus, the term “derivatives” insofar as it modifies the hydroxyacids, preferably denotes these compounds. Suitable examples include, but are not limited to glycolic acid methyl ester and ethyl ester, O-acetyl-mandelic acid and O-acetyl-benzilic acid. The hydroxyacids can be used as free acid, amide, lactone, ester or salt in full or partial form. Those skilled in the art will appreciate the myriad derivatives of hydroxyacids, given the description of the various hydroxyacids herein, as well as the definition of derivative above.
(5) Related Hydroxycarboxylic Acids

The expression “related hydroxycarboxylic acids” denotes those hydroxyacids in which the hydroxyl group is at any carbon position other than the alpha position, or the hydroxyl group is replaced by a keto group, or other miscellaneous organic hydroxycarboxylic acids that are not readily represented by a generic structure. For convenience this group of compounds can be subdivided into (a) alpha ketoacids and (b) miscellaneous hydroxyacids.
(a) Alpha Ketoacids

Ketoacids are related to hydroxyacids in that the hydroxyl group is replaced by the keto group. Although the keto group can be at any position other than the terminal ends, the preferred keto group is an alpha ketoacid. For example pyruvic acid, an alpha ketoacid is related to lactic acid in that the hydroxyl group of lactic acid is substituted by a keto group. In the skin, the lactate dehydrogenase enzyme is believed to convert pyruvate to lactate and vice visa. The ketoacids have been found to have similar therapeutic effects as that of alpha hydroxyacids. The generic structure of alpha ketoacids may be represented as follows:
(Ra)COCOOH
wherein Ra is H, alkyl, aralkyl or aryl group of saturated or unsaturated, isomeric or non-isomeric, straight or branched chain or cyclic form, having 1 to 25 carbon atoms, and in addition Ra may carry F, Cl, Br, I, OH, CHO, COOH and alkoxyl group having 1 to 9 carbon atoms. The typical alkyl, aralkyl, aryl and alkoxyl groups for Ra include methyl, ethyl, propyl, isopropyl, butyl, pentyl, octyl, lauryl, stearyl, benzyl, phenyl, methoxyl and ethoxyl.

Representative alpha ketoacids that may be useful in embodiments are listed below: 2-ketoethanoic acid (glyoxylic acid), 2-ketopropanoic acid (pyruvic acid), 2-phenyl-2-ketoethanoic acid (benzoylformic acid), 3-phenyl-2-ketopropanoic acid (phenylpyruvic acid), 2-ketobutanoic acid, 2-ketopentanoic acid, 2-ketohexanoic acid, 2-ketoheptanoic acid, 2-ketooctanoic acid and 2-ketododecanoic acid.
(b) Miscellaneous Hydroxyacids

These hydroxyacids have similar therapeutic effects as the effects of alpha hydroxyacids but their chemical structures are not readily represented by the foregoing generic structures. The expression “miscellaneous hydroxyacids” therefore will denote at least one of the compounds listed below: agaricic acid, aleuritic acid, citramalic acid, glucosaminic acid, galactosaminic acid, 2-keto-gulonic acid and 2-keto-gulonolactone, mannosaminic acid, mevalonic acid and mevalonolactone, pantoic acid and pantolactone, quinic acid (1,3,4,5-tetrahydroxycyclohexanecarboxylic acid), piscidic acid (4-hydroxybenzyltartaric acid), isoascorbic acid (D-erythro-hex-2-enonic acidr-lactone), 2-hexulosonic acids (isomers; arabino-2-hexulosonicacid, xylo-2-hexulosonic acid, ribo-2-hexulosonic acid, lyxo-2-hexulosonic acid), 5-hexulosonic acids (isomers; arabino-5-hexulosonic acid, xylo-5-hexulosonic acid, ribo-5-hexulosonic acid, lyxo-5-hexulosonic acid).

(B) N-Acyl-Aldosamines

N-Acyl-aldosamines preferably include, but are not limited to, N-acylated aldosamines in which the acylamino group is located at any carbon position except the carbon one position. In accordance with the present invention, the generic structure or formula of N-acyl-aldosamines can be represented as follows:
R₁—(CHOH)ₘ—CH(NHCOR₂)—(CHOH)ₙ—R₃
where R₁ is selected from the group consisting of H, COOH, an alkyl, alkoxyl, aralkyl or aryl group having 1 to 19 carbon atoms; R₂ is selected from the group consisting of an alkyl, aralkyl or aryl group having 1 to 19 carbon atoms; m, n is independently from 0-19; R₃ is selected from the group consisting of CHO, CONH₂, and COOR₄; and R₄ is selected from the group consisting of H, an alkyl, aralkyl or aryl group having 1 to 9 carbon atoms; H attached to a carbon atom may be substituted by I, F, Cl, Br, or an alkyl, alkoxyl, aralkyl or aryl group having 1 to 19 carbon atoms. N-Acyl-aldosamines can be present as saturated or unsaturated, stereoisomeric or non-stereoisomeric, straight or branched chain or cyclic form. A typical cyclic form of an N-acyl-aldosamine is a five member ring (furanose form) or a six member ring (pyranose form).

The following are representative N-acyl-aldosamines.
(1) N-Acetyl-aldosamines.
   N-acetyl-glycerosamine, N-acetyl-erythrosamine, N-acetyl-threosamine, N-acetyl-ribosamine, N-Acetyl-arabinosamine, N-Acetyl-xylosamine, N-Acetyl-lyxosamine, N-Acetyl-allosamine, N-Acetyl-altrosamine, N-Acetyl-glucosamine, N-Acetyl-mannosamine, N-Acetyl-gulosamine, N-Acetyl-idosamine, N-Acetyl-galactosamine, N-Acetyl-talosamine, N-Acetyl-glucoheptosamine, N-Acetyl-galactoheptosamine, N-Acetyl-mannoheptosamine, N-acetyl-lactosamine, N-acetyl-muramic acid, N-acetyl-neuramine, N-acetyl-neuramin lactose, N-acetyl-glyceraminic acid, N-acetyl-erythrosaminic acid, N-acetyl-threosaminic acid, N-acetyl-ribosaminic acid, N-acetyl-arabinosaminic acid, N-acetyl-xylosaminic acid, N-acetyl-lyxosaminic acid, N-acetyl-allosaminic acid, N-acetyl-altrosaminic acid, N-acetyl-glucosaminic acid, N-acetyl-mannosaminic acid, N-acetyl-gulosaminic acid, N-acetyl-idosaminic acid, N-acetyl-galactosaminic acid, N-acetyl-talosaminic acid, N-acetyl-heptoglucosaminic acid, N-acetyl-heptogalactosaminic acid, N-acetyl-heptomannosaminic acid, N-acetyl-neuraminic acid (N-acetyl-nonulosaminic acid), N-acetyl-5-amino-5-deoxynonose), N-acetyl-hyalobiouronic acid, N-acetyl-chondrosine, N-acetyl-streptomycin, N-acetyl-erythromycin, N-acetyl-gentamycin, N-acetyl-nojirimycin, N-acetyl-glucosylamine, N-acetyl-mannosylamine and N-acetyl-galactosylamine.

Examples of five and six member ring forms are

2-acetamido-2-deoxy-D-ribofuranoside, 2-acetamido-2-deoxy-D-ribopyranoside, 2-acetamido-2-deoxy-D-glucofuranoside, 2-acetamido-2-deoxy-D-glucopyranoside, 2-acetamido-2-deoxy-D-galactofuranoside and 2-acetamido-2-deoxy-D-galactopyranoside.

2) N-Propanoyl-aldosamines.

N-propanoyl-glycerosamine, N-propanoyl-erythrosamine, N-propanoyl-threosamine, N-propanoyl-ribosamine, N-Propanoyl-arabinosamine, N-Propanoyl-xylosamine, N-Propanoyl-lyxosamine, N-Propanoyl-allosamine, N-Propanoyl-altrosamine, N-Propanoyl-glucosamine (N-propanoyl-chitosamine), N-Propanoyl-mannosamine, N-Propanoyl-gulosamine, N-Propanoyl-idosamine, N-Propanoyl-galactosamine, N-Propanoyl-talosamine, N-Propanoyl-glucoheptosamine, N-Propanoyl-galactoheptosamine, N-Propanoyl-mannoheptosamine, N-propanoyl-lactosamine, N-propanoyl-muramic acid, N-propanoyl-neuramine, N-propanoyl-neuramin lactose, N-propanoyl-glyceraminic acid, N-propanoyl-erythrosaminic acid, N-propanoyl-threosaminic acid, N-propanoyl-ribosaminic acid, N-propanoyl-arabinosaminic acid, N-propanoyl-xylosaminic acid, N-propanoyl-lyxosaminic acid, N-propanoyl-allosaminic acid, N-propanoyl-altrosaminic acid, N-propanoyl-glucosaminic acid, N-propanoyl-mannosaminic acid, N-propanoyl-gulosaminic acid, N-propanoyl-idosaminic acid, N-propanoyl-galactosaminic acid, N-propanoyl-talosaminic acid, N-propanoyl-heptoglucosaminic acid, N-propanoyl-heptogalactosaminic acid, N-propanoyl-heptomannosaminic acid, N-propanoyl-neuraminic acid (N-propanoyl-nonulosaminic acid), N-propanoyl-5-amino-5-deoxynonose), N-propanoyl-hyalobiouronic acid, N-propanoyl-chondrosine, N-propanoyl-streptomycin, N-propanoyl-erythromycin, N-propanoyl-gentamycin, N-propanoyl-nojirimycin, N-propanoyl-glucosylamine, N-propanoyl-mannosylamine, N-propanoyl-galactosylamine, and other N-propanoyl-glycosylamines.

(C) N-Acylamino Acids and Related Compounds.

N-Acylamino acids preferably are N-acyl derivatives of amino acids. In accordance with the embodiments, the generic structure or formula of N-acylamino acids can be represented as follows:
R₁—CH(NHCOR₂)—(CH₂)ₙ—COR₃
where R₁ is H, an alkyl, aralkyl or aryl group having 1 to 18 carbon atoms; R₂ is an alkyl, aralkyl or aryl group having 1 to 18 carbon atoms; n is an integer, preferably from 0 to 5; R₃ is OH, NH₂ or OR₃; and R₃ is an alkyl, aralkyl or aryl group having 1 to 9 carbon atoms; and in addition R₁ may carry OH, SH, SCH₃, COOH, NH₂, CONH₂, NHCONH₂, NHC(═NH)NH₂, imidazole, pyrrolidine or other heterocyclic group; the H attached to a carbon atom may be substituted by I, F, Cl, Br, OH or alkoxyl group having 1 to 9 carbons. N-Acylamino acids can be present as saturated or unsaturated, stereoisomeric or non-stereoisomeric, straight or branched chain or cyclic form, as a free acid, salt or partial salt with organic or inorganic alkali, amide, ester or lactone form. Among commonly known N-acylamino acids and derivatives, N-acyl-proline and its derivatives cannot be represented by the above structure because the alpha amino group is part of the heterocyclic pyrrolidine ring.

The following are representative N-acylamino acids and related compounds.
(1) N-Acetylamino Acids

N-acetyl-alanine, N-acetyl-arginine, N-acetyl-asparagine, N-acetyl-aspartic acid, N-acetyl-cysteine, N-acetyl-glycine, N-acetyl-glutamic acid, N-acetyl-glutamine, N-acetyl- histidine, N-acetyl-isoleucine, N-acetyl-leucine, N-acetyl-lysine, N-acetyl-methionine, N-acetyl-phenylalanine, N-acetyl-proline, N-acetyl-serine, N-acetyl-threonine, N-acetyl-tryptophan, N-acetyl-tyrosine and N-acetyl-valine.
(2) Related N-Acetylamino Acids:

These may or may not be represented by the above generic structure. Throughout this description, the expression “related N-Acetylamino acids” denotes one or more of the following: N-acetyl-β-alanine, N-acetyl-γ-aminobutanoic acid, N-acetyl-β-aminoisobutanoic acid, N-acetyl-citrulline, N-acetyl-dopa (N-acetyl-3,4-dihydroxyphenylalanine), N-acetyl-homocysteine, N-acetyl-homoserine, N-acetyl-ornithine, N-acetyl-phenylglycine, N-acetyl-4-hydroxyphenylglycine and N,O-diacetyl-4-hydroxyphenylglycine.

The above N-acetylamino acids and related compounds can be present as a free acid, salt or partial salt with organic or inorganic alkali, lactone, amide, ester or stereoisomeric form. As an illustration, N-acetyl-proline includes for example, N-acetyl-L-proline, N-acetyl-L-proline sodium salt, N-acetyl-L-prolinamide, N-acetyl-L-proline methyl ester, N-acetyl-L-proline ethyl ester, N-acetyl-L-proline propyl ester and N-acetyl-L-proline isopropyl ester.
(3) N-Propanoylamino Acids.

Suitable N-propanoylamino acids include, but are not limited to, N-propanoyl-alanine, N-propanoyl-arginine, N-propanoyl-asparagine, N-propanoyl-aspartic acid, N-propanoyl-cysteine, N-propanoyl-glycine, N-propanoyl-glutamic acid, N-propanoyl-glutamine, N-propanoyl- histidine, N-propanoyl-isoleucine, N-propanoyl-leucine, N-propanoyl-lysine, N-propanoyl-methionine, N-propanoyl-phenylalanine, N-propanoyl-proline, N-propanoyl-serine, N-propanoyl-threonine, N-propanoyl-tryptophan, N-propanoyl-tyrosine and N-propanoyl-valine.
(4) Related N-Propanoylamino Acids:

These may or may not be represented by the above generic structure. Throughout this description, the expression “related N-propanoylamino acids” denotes one or more of the following: N-propanoyl-β-alanine, N-propanoyl-γ-aminobutanoic acid, N-propanoyl-β-aminoisobutanoic acid, N-propanoyl-citrulline, N-propanoyl-dopa (N-propanoyl-3,4-dihydroxyphenylalanine), N-propanoyl-homocysteine, N-propanoyl-homoserine, N-propanoyl-ornithine, N-propanoyl-phenylglycine, N-propanoyl-4-hydroxyphenylglycine and N,O-dipropanoyl-4-hydroxyphenylglycine.

The above N-propanoylamino acids and related compounds can be present as a free acid, salt or partial salt with organic or inorganic alkali, lactone, amide, ester or stereoisomeric form. As an illustration, N-propanoyl-proline includes for example, N-propanoyl-L-proline, N-propanoyl-L-proline sodium salt, N-propanoyl-L-prolinamide, N-propanoyl-L-proline methyl ester, N-propanoyl-L-proline ethyl ester, N-propanoyl-L-proline propyl ester and N-propanoyl-L-proline isopropyl ester.

Embodiments described herein include a composition that comprises one or more of the above-described compounds in a therapeutically or cosmetically effective amount to provide at least one of the following effects: plump and pout lips; enhance and firm eyelids; enlarge and augment breasts; and elongate and expand the penis.

Embodiments of the invention also include a method of plumping and pouting lips, a method of enhancing and firming eyelids, a method of enlarging and augmenting breasts, and a method of elongating and expanding the penis comprising administering the affected area a composition comprising at least one selected from the group consisting of a hydroxycarboxylic acid, N-acyl-aldosamine, N-acylamino acid or related compounds, in an effective amount and for an effective period of time to achieve the desired effect.

The effective period of time for administering the composition of embodiments described herein will vary on the area to be effected, the desired effect, the weight of the patient, and the degree of effect desired. Typically, the composition is applied twice daily for at least two weeks. Preferably, the composition is applied to the affected area twice daily for at least one month, more preferably at least two months, and even more preferably, at least three months, and even more preferably, at least 6 months. The composition preferably is applied to the following areas: (i) lips to provide plump and pouting lips; (ii) eyelids to enhance and firm the eyelids; (iii) breasts to enlarge and augment the breasts; and (iv) penis to elongate and expand the penis. Using the guidelines provided herein, those skilled in the art will be capable of determining the effective amount and effective period of time, depending on the subject and the desired treatment.

### Measurements and Test Results

There were two different approaches to determine if a test substance has any effects on the plumping and pouting of lips, enhancing and firming of eyelids, enlarging and augmenting breasts, elongating and expanding penis. The first and the easiest approach is to measure if a test substance has any effects on the plumping or increasing the skin thickness of other body areas after topical administration. The second approach is a direct measurement before and after topical application to lips, eyelids, breasts and penis.

Skin Thickness Measurement

Measuring the increase in thickness of the skin was accomplished in accordance with the following procedures. The increased plumpness or skin thickness by topical application of active ingredients of the embodiments is mainly due to increased biosynthesis of dermal components in the skin. The three most important dermal components are glycosaminoglycans (GAGs), collagens and elastic fibers. The increased skin thickness caused by increased biosynthesis of dermal components can be distinguished from that by water retention or edema formation. In the former case, the skin remains thickened for weeks and months after termination of topical application. In the latter case, the increased skin thickness returns to the original skin thickness within days after termination of topical application.
(1) Forearm Skin

The most convenient body area for testing skin thickness is the forearm skin. For example, a test substance in a formulation was topically applied once or twice daily to left or right forearm for 3 to 6 months and the other forearm was untreated or treated with a vehicle control. The skin plumpness or thickness was measured by micrometer calipers as follows. The skin was grasped with a 2×6 cm hinge plate, the internal faces of the hinge were coated with soft cloth to prevent slippage, and manually squeezed to a threshold point when the subject felt tight. Combined skin thickness of two whole-skin layers including thickness of the two hinge leaves was measured with micrometer calipers. Thickness of the two hinge leaves was subtracted to determine the actual thickness of two whole-skin layers. Triplicate measurements on treated site were done and an average number was used for calculation of the skin thickness.

The skin thickness also can be measured by Micro Image Analysis System. Upon completion of topical application, punch biopsy specimens were taken from the treated skin and the untreated skin or treated with vehicle control, and were placed immediately into the fixative solution, and processed for histochemical staining and analysis. The thickness of the skin was measured by Micro Image Analysis System on papillary dermis, and the mean thickness was expressed as area of epidermis/horizontal length.

Hydroxycarboxylic acids, N-acyl-aldosamines, N-acylamino acids and related compounds at various concentrations were topically applied to forearms of volunteer subjects or patients. As shown in Tables 1-7, the skin plumpness or thickness was increased substantially by the compounds of the present invention. The increased skin thickness was believed to be due to increased biosynthesis of GAGs, collagen and elastic fibers. Hydroxycarboxylic acids, N-acyl-aldosamines, N-acylamino acids and related compounds also can be incorporated as a combination with two or more active ingredients in the same formulation to provide synergetic or synergistic effects on the skin as shown in Table 5. From the above test results, hydroxycarboxylic acids, N-acyl-aldosamines, N-acylamino acids and related compounds used alone or in combination should be beneficial to plump and pout lips, enhance and firm eyelids, enlarge and augment breasts, elongate and expand penis.

Using the same test conditions, many compounds were tested that did not fall within the context of any of the above-defined compounds that are useful in the embodiments described herein. These compounds did not increase the skin thickness after topical application. For example, salicylic acid (2-hydroxybenzoic acid), a typical keratolytic agent, is used for topical treatment of acne and hyperkeratotic conditions such as calluses and dry skin. Salicylic acid does not fall within the context of any of the above-defined compounds that are useful in the embodiments described herein because the hydroxyl group is directly attached to the benzene ring and the hydroxyl group is acidic instead of neutral in chemical property as described in the earlier definition of the hydroxycarboxylic acids. Under the same test conditions, salicylic acid at 5% concentration decreased instead of increasing skin thickness as shown in Table 8. It is expected that salicylic acid is not a candidate for topical treatment to enlarge breasts, plump lips, firm eyelids or elongate penis.

TABLE 1
Alpha-Hydroxyacids (AHAs) Increased Skin Thickness

| | Subject | | |
|---|---|---|---|
| | (Age & | Duration^{a} | % Increase |
| AHA | sex) | (Months) | Over Control^{b} |
| Glycolic Acid 10% Lotion | 73M | 5 | 11 |
| | 58F | 8 | 22 |
| | 77F | 4 | 30 |
| | 59F | 4 | 43 |
| DL-Lactic Acid 10% Lotion | 69F | 5 | 17 |
| | 59F | 6 | 31 |
| | 69M | 7 | 34 |
| | 70M | 5 | 42 |
| Methyllactic Acid 20% Lotion | 76F | 1 | 14 |
| | 67F | 2 | 16 |
| | 65F | 3 | 20 |
| DL-Mandelic Acid 30% Solution | 55F | 4 | 22 |
| | 62F | 4 | 27 |
| Benzilic Acid 35% Solution | 68F | 6 | 22 |
| | 72M | 6 | 45 |

^{a}twice daily topical application on forearm skin
^{b}opposite forearm

TABLE 2
Citric Acid (which is an AHA & a BHA) Increased Skin
Thickness

| Subject | Duration^{a} | Percentage Increase |
|---|---|---|
| (Age & sex) | (Month) | Over Control (%)^{b} |
| 57F | 8 | 7 |
| 53F | 6 | 8 |
| 52F | 6 | 9 |
| 74F | 6 | 11 |
| 75F | 5 | 13 |
| 72F | 7 | 16 |
| 50F | 9 | 19 |
| 51F | 8 | 19 |
| 76F | 5 | 23 |
| 75F | 5 | 26 |
| 75F | 5 | 27 |
| 70F | 5 | 41 |
| 83M | 5 | 55 |

^{a}20-25% lotion twice daily topical application on forearm skin
^{b}opposite forearm

TABLE 3
Polyhydroxy Acid (PHA) and Aldobionic Acid (ABA)
Increased Skin Thickness

| | Conc. | Subject | Duration^{a} | % Increase |
|---|---|---|---|---|
| PHA/ABA | (%) | (Age & sex) | (Months) | Over Control (%)^{b} |
| D-Gluconolactone | 20 | 62F | 2 | 7 |
| | 20 | 70F | 3 | 12 |
| | 20 | 79F | 2 | 13 |
| | 20 | 81F | 7 | 17 |
| | 20 | 79F | 4 | 18 |
| | 20 | 72F | 2 | 19 |
| Lactobionic Acid | 10 | 63F | 4 | 5 |
| | 20 | 61F | 4 | 10 |
| | 22 | 56F | 4 | 12 |
| | 10 | 64F | 5 | 12 |
| | 22 | 56F | 8 | 13 |
| | 8 | 76M | 3 | 26 |
| | 22 | 49F | 10 | 58 |

^{a}twice daily topical application on forearm skin
^{b}opposite forearm

TABLE 4
Summary of Increased Skin Thickness by
Hydroxycarboxylic Acid^{a}

| Substance | Subject | | Duration^{b} | % Increase |
|---|---|---|---|---|
| Control | (Number) | Age Range | (Months) | Over |
| Benzilic Acid | 2 | 68-72 | 2 | 22-45 |
| Citric Acid | 13 | 50-83 | 5-9 | 7-55 |
| Glycolic Acid | 4 | 58-77 | 4-8 | 11-43 |
| Gluconolactone | 6 | 62-81 | 2-7 | 7-19 |
| Lactic Acid | 4 | 59-70 | 5-7 | 17-42 |
| Lactobionic Acid | 7 | 49-76 | 1-3 | 5-58 |
| Mandelic Acid | 2 | 55-62 | 1 | 22-27 |
| Methyllactic Acid | 3 | 65-76 | 1-3 | 14-20 |

^{a}10-35% Concentration twice daily on forearm skin

TABLE 5
N-Acyl-aldosamines Increased Skin Thickness

| | | | % Increase |
|---|---|---|---|
| | Subject | Duration^{a} | Over |
| N-Acyl-aldosamines | (Age & sex) | (Weeks) | Control (%)^{b} |
| N-Acetyl-D-glucosamine | 56F | 3 | 11 |
| (10% lotion) | 74F | 3 | 25 |
| | 72F | 3 | 21 |
| N-Acetyl-D-glucosamine | 72F | 3 | 78 |
| 10% and | 74F | 3 | 26 |
| D-Gluconolactone 10% cream | | | |
| N-Propanoyl-D-glucosamine | 45F | 5 | 19 |
| 10% Solution | | 8 | 33 |
| | 85F | 4 | 13 |
| | | 6 | 27 |
| | 63F | 4 | 8 |
| | | 6 | 23 |
| | | 9 | 28 |

^{a}twice daily topical application on forearm skin
^{b}opposite forearm

TABLE 6
N-Acetylamino Acids Increased Skin Thickness

| | | | % Increase |
|---|---|---|---|
| | Subject | Duration^{a} | Over |
| N-Acetylamino Acids | (Age & sex) | (Weeks) | Control (%)^{b} |
| N-Acetyl-L-cysteine | 71F | 3 | 11 |
| 5% Lotion | 66F | 5 | 18 |
| | 56F | 3 | 11 |
| N-Acetyl-L-cysteine Methyl | 72F | 3 | 21 |
| Ester | 59F | 3 | 11 |
| 10% Lotion | 76F | 3 | 63 |
| 5% Lotion | | | |
| N-Acetyl-DL-homocysteine | 59F | 3 | 13 |
| Thiolactone | 76M | 3 | 65 |
| 5% Lotion | | | |
| N-Acetyl-L-tyrosine Ethyl Ester | 72F | 4 | 34 |
| 10% Solution | 47F | 4 | 11 |
| N-Acetyl-DL-tryptophan | 71F | 3 | 9 |
| 10% Lotion | 56F | 3 | 17 |
| | 71F | 3 | 14 |
| | 66F | 5 | 10 |
| Nα-Acetyl-L-arginine | 72F | 4 | 22 |
| 10% Solution | 47F | 4 | 32 |

^{a}twice daily topical application on forearm skin
^{b}opposite forearm

TABLE 7
N-Acetyl-L-proline 8% Solution Increased Skin Thickness

| Subject | Duration^{a} | Percentage Increase |
|---|---|---|
| (Age & sex) | (Week) | Over Control (%)^{b} |
| 55F | 5 | 7 |
| 46F | 4 | 8 |
| 64F | 5 | 11 |
| 67F | 3 | 17 |
| 61F | 6 | 17 |
| 72F | 3 | 25 |
| 49F | 4 | 27 |
| 72F | 6 | 50 |

^{a}once to twice daily topical application on forearm skin
^{b}opposite forearm

TABLE 8
Decreased Skin Thickness by Salicylic Acid

| | Duration^{b} | % Decrease |
|---|---|---|
| Subject^{a} | (Week) | Over Control (%) |
| 57F | 8 | −6 |
| 60F | 3 | −7 |
| 59F | 6 | −8 |
| 63F | 10 | −11 |
| 61F | 2 | −12 |
| 60F | 5 | −14 |
| 67F | 6 | −21 |
| 49F | 3 | −23 |
| 73F | 7 | −32 |

^{a}Age and sex
^{b}5% solution twice daily topical application on forearm skin

(2) Eyelids Skin

A plumping or firming effect on eyelids skin by topical application of active ingredients of the embodiments described herein also is an indication for beneficial effects to plump and pout lips, enhance and firm eyelids, enlarge and augment breasts, and elongate and expand penis.

Compared with other body skin, the eyelids are the thinnest skin, approximately 0.4 mm in skin thickness. A test substance at 5-10% concentration in a solution or emulsion was topically applied once or twice daily for one to six months. Standard photographs were taken before and after topical applications of test formulations. Plumping or firming effect was determined by clinical as well as photographic evaluation. It was discovered that hydroxycarboxylic acids, N-acyl-aldosamines, N-acylamino acids and related compounds had substantial effect on plumping or firming the eyelids.

Aside from clinical as well as photographic evaluation, eyelids plumping or firming also was determined from the disappearance of certain skin lines on the eyelids skin. When eyelids skin was plumping up, the eyelids skin firmed up or pulled up, and the skin lines became less apparent or disappeared. Therefore, when the skin lines on the eyelids disappeared after topical application of a test formulation, the test substance was judged to have plumping or firming effect on the eyelids. It also was discovered that hydroxycarboxylic acids, N-acyl-aldosamines, N-acylamino acids and related compounds on topical application had firming or plumping up effects on the eyelids skin. The results showed that hydroxycarboxylic acids, N-acyl-aldosamines, N-acylamino acids and related compounds should be therapeutically beneficial for topical administration to plump and pout lips, enhance and firm eyelids, enlarge and augment breasts, elongate and expand penis.
(B) Enlargement, Plumping and Elongation

A test formulation containing a hydroxycarboxylic acid, N-acyl-aldosamine, N-acylamino acid or related compound was topically applied once or twice daily to skin or mucus membrane organs or sites such as lips, eyelids, breasts and penis for one to six months. Increased fullness, volume, contour or the effect of enlarging, expanding, augmenting, enhancing, firming, pouting, plumping or elongating after topical application was measured or determined by clinical as well as photographic evaluations. One of the measurements for breast enlargement or augmentation was by the size of brassiere cup. After topical application of the active ingredient, the subject would feel fullness or greater tightness of her treated breast with increased volume and contour, and there was a need to change and increase the size of the brassiere cup. Thus the enlargement or augmentation of the breast was determined by clinical as well as the increased size of brassiere cup.

The mammary glands, especially female breasts are composed of fat, muscle, glandular tissue and dermal components which include GAGs, collagen fibers and elastic fibers. Although the amount of fat tissue around the lobes of glandular tissue usually determines the size of the breasts, the increased biosynthesis of dermal components can also contribute to the enlargement of the breasts. Since topical application of an hydroxycarboxylic acid, N-acyl-aldosamine or N-acylamino acid has been shown to plump the skin and to increase the skin thickness by stimulating biosynthesis of dermal components, the enlargement and augmentation of the breasts could be due to increased amounts of dermal components and the fat.

The plumping, pouting or firming effects on lips and eyelids by topical application of an hydroxycarboxylic acid, N-acyl-aldosamine, N-acylamino acid or related compound was determined by clinical as well as photographic evaluation. The elongation or expanding effect of penis was determined by direct measurement of penis before and after once or twice daily topical application of an hydroxycarboxylic acid, N-acyl-aldosamine, N-acylamino acid or related compound.
(C) Antioxidants

Breast tumors and cancers include fibrocystic tumor, fibroadenoma and carcinomas. Since most tumor and cancer formation involves free radicals and superoxides, antioxidant substances should help suppress or prevent the formation of breast tumors and cancers by interrupting or quenching the process of carcinogenesis.

An antioxidant can be defined as a substance capable of preventing or inhibiting oxidation. The antioxidant property can be readily determined by using any one of the following test methods: prevention or retardation of air oxidation of (a) anthralin, (b) hydroquinone, or (c) banana peel. A freshly prepared anthralin solution or cream is bright yellow, and an air oxidized one is brownish or black. A hydroquinone solution or cream is colorless or white color, and an air oxidized one is brownish or black. A freshly peeled banana peel is light yellow in color and an oxidized one ranges in color from tan, dark tan, brown to brownish black.

For example, in control experiments, fresh banana peels cut into sizes of 1×2 cm in 50 mm plastic petri dishes containing 5 ml water at neutral or acidic pH changed in color from white-yellowish to tan within 6 hours at room temperature, and changed to dark tan color during the next period of 24 to 72 hours. When fresh banana peels were placed in dishes containing 5 ml of 0.1 M lactobionic acid under the same conditions, the banana peels remained white-yellowish for the period of 24 hours, and changed in color to tan after 72 hours. The above test results show that lactobionic acid is an antioxidant substance. Using anthralin and hydroquinone test methods also confirmed that lactobionic acid is a moderate antioxidant. Many hydroxycarboxylic acids including all the PHAs and ABAs, N-acyl-aldosamines, N-acylamino acids and related compounds also have been shown to have antioxidant properties, based on the above screen tests. The antioxidant substances of certain embodiments of the invention include, for example but not limited to, citric acid, isocitric acid, malic acid, tartaric acid, pantolactone, isoascorbic acid, polyhydroxy acids, aldobionic acids and N-acetyl-cysteine. These antioxidant substances should be therapeutically beneficial for topical application to prevent breast tumors and cancers.

### Synergetic and Synergistic Compositions

A cosmetic, pharmaceutical or other topical agent also can be added or incorporated into a composition comprising hydroxycarboxylic acids, N-acyl-aldosamines, N-acylamino acids and/or related compounds of the embodiments to exert synergetic or synergistic effects. The topical agent preferably is selected from the group consisting of agents that improve or eradicate age spots, keratoses and wrinkles; local analgesics and anesthetics; antiacne agents; antibacterials; antiyeast agents; antifungal agents; antiviral agents; antidermatitis agents; antihistamine agents; antipruritic agents; antiinflammatory agents; antipsoriatic agents; antiseborrheic agents; antiaging and antiwrinkle agents; sunblock and sunscreen agents; skin lightening agents; depigmenting agents; vitamins; corticosteroids; tanning agents; humectants; estrogens; androgens; hormones and retinoids.

One or more than one topical agent preferably is selected from the group consisting of aclovate, acyclovir, acetylsalicylic acid, adapalene, albuterol, aluminum acetate, aluminum chloride, aluminum hydroxide, aluminum chlorohydroxide, amantadine, aminacrine, aminobenzoic acid (PABA), aminocaproic acid, aminosalicylic acid, amitriptyline, anthralin, ascorbic acid, ascoryl palimate, atropine, azelaic acid, bacitracin, bemegride, beclomethasone dipropionate, benzophenone, benzoyl peroxide, betamethasone dipropionate, betamethasone valerate, brompheniramine, bupivacaine, butoconazole, calcipotriene, camphor, capsaicin, carbamide peroxide, chitosan, chlorhexidine, chloroxylenol, chlorpheniramine, ciclopirox, clemastine, clindamycin, clioquinol, clobetasol propionate, clotrimazole, coal tar, cromolyn, crotamiton, cycloserine, dehydroepiandrosterone, desoximetasone, dexamethasone, diphenhydramine, doxypin, doxylamine, dyclonine, econazole, erythromycin, estradiol, estrone, ethinyl estradiol, fluocinonide, fluocinolone acetonide, 5-fluorouracil, griseofulvin, guaifenesin, haloprogin, hexylresorcinol, homosalate, hydrocortisone, hydrocortisone 21-acetate, hydrocortisone 17-valerate, hydrocortisone 17-butyrate, hydrogen peroxide, hydroquinone, hydroquinone monoether, hydroxyzine, ibuprofen, ichthammol, imiquimod, indomethacin, ketoconazole, ketoprofen, kojic acid, lidocaine, meclizine, meclocycline, menthol, mepivacaine, methyl nicotinate, metronidazole, miconazole, minocycline, minoxidil, monobenzone, mupirocin, naftifine, naproxen, neomycin, nystatin, octyl methoxycinnamate, octyl salicylate, oxybenzone, oxiconazole, oxymetazoline, padimate O, permethrin, pheniramine, phenol, phenylephrine, phenylpropanolamine, piperonyl butoxide, podophyllin, podofilox, povidone iodine, pramoxine, prilocaine, procaine, promethazine propionate, propranolol, pseudoephedrine, pyrethrin, pyrilamine, resorcinol, retinal, 13-cis retinoic acid, retinoic acid, retinol, retinyl acetate, retinyl palmitate, selenium sulfide, shale tar, sulconazole, sulfur, sulfadiazine, tazarotene, testosterone, terbinafine, terconazole, tetracaine, tetracycline, tetrahydrozoline, thymol, tioconazole, tolnaftate, triamcinolone diacetate, triamcinolone acetonide, triamcinolone hexacetonide, triclosan, triprolidine, undecylenic acid, urea, vitamin E acetate, wood tar and zinc pyrithione.

### General Preparations

Compositions comprising hydroxycarboxylic acids, N-acyl-aldosamines, N-acylamino acids and/or related compounds of the embodiments described herein can be formulated as a solution, a gel, a lotion, a cream, an ointment, a spray, a stick, a powder, a masque, a rinse, a wash or other form acceptable for topical use on the skin or mucocutaneous organs or sites.

To prepare a solution composition, at least one hydroxycarboxylic acid, N-acyl-aldosamine, N-acylamino acid, related compound or in combination with one another in an effective amount preferably is dissolved in a solution prepared from water, ethanol, propylene glycol, butylene glycol, and/or other topically acceptable vehicle. The effective amount of the particular compound will vary on the area to be effected, the desired effect, the weight of the patient, and the degree of effect desired. The effective amount (e.g., concentration) of a single active ingredient or the total concentration of all the active ingredients can range from 0.01 to 99.9% by weight of the total composition, with preferred concentration of from 0.1 to 50% by weight of the total composition and with more preferred concentration of from 1 to 25% by weight of the total composition, and more preferably from about 1 to about 20% by weight, based on the total weight of the composition.

To prepare a topical composition in lotion, cream or ointment form, a hydroxycarboxylic acid, N-acyl-aldosamine, N-acylamino acid, related compound or in combination with one another preferably is first dissolved in water, ethanol, propylene glycol, and/or other vehicle, and the solution thus obtained is mixed with a desired base or pharmaceutically acceptable vehicle to make lotion, cream or ointment. Concentrations of the active ingredients are the same as described above.

A topical composition of the instant invention can also be formulated in a gel form. A typical gel composition preferably is formulated by the addition of a gelling agent such as chitosan, methyl cellulose, ethyl cellulose, polyvinyl alcohol, polyquaterniums, hydroxyethylcellulose, hydroxypropylcellulose, hydroxypropylmethylcellulose, carbomer or ammoniated glycyrrhizinate to a solution comprising the hydroxycarboxylic acid or its derivative. The preferred concentration of the gelling agent may range from 0.1 to 4 percent by weight of the total composition. Concentrations of the active ingredients are the same as described above.

To prepare a combination composition for synergetic or synergistic effects, a cosmetic, pharmaceutical or other topical agent preferably is incorporated into any one of the above compositions by dissolving or mixing the agent into the formulation.

A composition containing a hydroxycarboxylic acid, N-acyl-aldosamine, N-acylamino acid and/or related compound with a free acid form usually has a pH below 2.0, and such composition can be used for topical application to skin or mucous membrane without stinging or irritation for most people. However, the composition may be stinging or irritating for some people with sensitive skin or mucous membrane on repeated topical application, due to lower pH or more importantly due to uncontrolled release and fast penetration of the active ingredient into the skin or mucous membrane. To prevent such instances, the composition can be partially neutralized with organic or inorganic alkali such as ammonium hydroxide, ethylenediamine, dimethylaminoethanol, 2-amino-2-methyl-1-propanol and sodium hydroxide. The organic or inorganic alkali preferably is first dissolved in water to make 1 to 5 N solution, and such alkaline solution is added to the composition containing the active ingredients of the present invention until the pH of the composition is raised to between 3.0 and 4.0.

To prepare a control-release composition in an amphoteric system or molecular complex, an amphoteric, pseudoamphoteric substance or complexing agent is first added to react with the active ingredients of the present invention to form ionic complex in amphoteric or pseudoamphoteric system. Amino acids and related compounds are suitable amphoteric or pseudoamphoteric substances, such as arginine, lysine, ornithine and creatinine.

Other forms of compositions for delivery of a hydroxycarboxylic acid, N-acyl-aldosamine, N-acylamino acid or related compound of the embodiments are readily blended, prepared or formulated by those skilled in the art.

The invention now will be explained with reference to the following non-limiting examples.

### EXAMPLE 1

In one of the studies, skin thickness was measured by micrometer calipers as follows: The skin was grasped with a 2×6 cm metal hinge; the internal faces of which were coated with emery cloth to prevent slippage, and manually squeezed to threshold subject discomfort. Combined thickness of two whole-skin layers including thickness of the two hinge leaves was measured with micrometer calipers. Thickness of the two hinge leaves was subtracted to determine the actual thickness of two whole-skin layers. Triplicate measurements on treated sites were conducted and an average number was used for calculation of the skin thickness.

### EXAMPLE 2

A typical N-propanoyl-aminocompound in a cream composition was formulated as follows: N-Propanoyl-proline 3 g was dissolved in warm water 9 ml and propylene glycol 3 ml, and the solution thus obtained was mixed uniformly with hydrophilic ointment 45 g. The cream thus formulated had pH 2.6 and contained 5% N-propanoyl-proline.

### EXAMPLE 3

N-Propanoyl-prolinamide 0.7 g was dissolved in warm water 2 ml and propylene glycol 1 ml, and the solution thus obtained was mixed uniformly with hydrophilic ointment 6.3 g. The cream thus formulated had pH 4.5 and contained 7% N-propanoyl-prolinamide.

### EXAMPLE 4

N-Propanoyl-tyrosine 3 g was dissolved in warm ethanol 5 ml and propylene glycol 13 ml, and the solution thus obtained was mixed uniformly with hydrophilic ointment 39 g. The cream thus formulated had pH 1,5 and contained 5% N-propanoyl-tyrosine.

### EXAMPLE 5

N-Propanoyl-methionine 3 g was dissolved in warm water 11 ml and propylene glycol 6 ml, and the solution thus obtained was mixed uniformly with hydrophilic ointment 40 g. The cream thus formulated had pH 2.2 and contained 5% N-propanoyl-methionine.

### EXAMPLE 6

N-Propanoyl-arginine 3 g was dissolved in warm water 7 ml and propylene glycol 5 ml, and the solution thus obtained was mixed uniformly with hydrophilic ointment 45 g. The cream thus formulated had pH 4.3 and contained 5% N-propanoyl-arginine.

### EXAMPLE 7

N-Propanoyl-glucosamine 10 g was dissolved in water 20 ml and propylene glycol 10 ml, and the solution thus obtained was mixed uniformly with hydrophilic ointment 60 g. The cream thus formulated had pH 5.3 and contained 10% N-Propanoyl-glucosamine.

### EXAMPLE 8

N-Propanoyl-glutamic acid 3g was dissolved in warm water 13 ml and propylene glycol 8 ml. Arginine 2 g was added to make an amphoteric system, and the solution thus obtained was mixed uniformly with hydrophilic ointment 34 g. The cream thus formulated had pH 5.1 and contained 5% N-propanoyl-glutamic acid in an amphoteric composition.

### EXAMPLE 9

N-Propanoyl-creatinine 3 g was dissolved in warm water 8 ml and propylene glycol 7 ml, and the solution thus obtained was mixed uniformly with hydrophilic ointment 42 g. The cream thus formulated had pH 4.8 and contained 5% N-propanoyl-creatinine.

### EXAMPLE 10

N-Propanoyl-prolinamide 1.5 g was dissolved in 30 ml solution prepared from water 40 parts, ethanol 40 parts and propylene glycol 20 parts by volume. The solution thus prepared had pH 4.9 and contained 5% N-propanoyl-prolinamide.

### EXAMPLE 11

N-Propanoyl-glucosamine 10 g was dissolved in 90 ml solution prepared from water 40 parts, ethanol 40 parts and propylene glycol 20 parts by volume. The formulation thus prepared had a pH 6.0, and contained 10% N-propanoyl-glucosamine in solution.

### EXAMPLE 12

A female subject, age 63, applied topically twice daily N-propanoyl-glucosamine 10% solution (EXAMPLE 11) to her left forearm for a total of 9 weeks. After 4 weeks there was no change in skin thickness of her untreated right forearm, her left forearm had increased 8% in skin thickness as measured by the micrometer calipers described above. After 6 weeks her left forearm had increased 23% and after 9 weeks 28% in skin thickness while there was no change in skin thickness of her right forearm. At the end of 9 weeks her untreated right forearm was still loose and relatively thin when lifted. In contrast, her left forearm was more firm, smooth and plump when lifted. This result indicated that N-propanoyl-glucosamine would be therapeutically effective for topical treatment to enlarge, plump or elongate mucocutaneous organs and sites.

### EXAMPLE 13

A female subject, age 45, applied topically twice daily N-propanoyl-glucosamine 10% solution (Example 11) to her left forearm for a total of 8 weeks. After 5 weeks there was no change in skin thickness of her untreated right forearm, her left forearm had increased 19% in skin thickness as measured by the micrometer calipers described above. After 8 weeks her left forearm had increased 33% in skin thickness. At the end of 8 weeks her untreated right forearm was still loose and relatively thin when lifted. In contrast, her left forearm was more firm, smooth and plump when lifted. This result indicated that N-propanoyl-glucosamine would be therapeutically effective for topical treatment to enlarge, plump or elongate mucocutaneous organs and sites.

### EXAMPLE 14

A female subject, age 85, applied topically twice daily N-propanoyl-glucosamine 10% solution (EXAMPLE 11) to her right forearm for a total of 6 weeks. After 4 weeks there was no change in skin thickness of her untreated left forearm, her right forearm had increased 13% in skin thickness as measured by the micrometer calipers described above. After 6 weeks her right forearm had increased 27% in skin thickness. At the end of 6 weeks her untreated left forearm was still loose and relatively thin when lifted. In contrast, her right forearm was more firm, smooth and plump when lifted. This result indicated that N-propanoyl-glucosamine would be therapeutically effective for topical treatment to enlarge, plump or elongate mucocutaneous organs and sites.

### EXAMPLE 15

A typical amphoteric composition containing a hydroxycarboxylic acid was formulated as follows. Glycolic acid 70% solution 10 g was dissolved in 10 ml water, and the solution thus obtained was mixed with oil-in-water base 80 g. The cream thus prepared had pH 1.8 and contained 7% glycolic acid. Alternatively, glycolic acid 70% solution 30 g was dissolved in water 20 ml and propylene glycol 20 ml. L-Arginine 2 g was added to the solution and the solution was mixed with oil-in-water base 138 g. The cream thus prepared had pH 2.1 and contained 10% glycolic acid.

### EXAMPLE 16

DL-Lactic acid 90% solution 10 g was dissolved in propylene glycol 10 ml and the solution thus prepared was mixed with oil-in-water base 80 g. The cream thus prepared had pH 1.9 and contained 9% DL-lactic acid.

### EXAMPLE 17

A typical polyhydroxy-lactone formulation was prepared as follows. Gluconolactone 24 g was dissolved in water 36 ml and propylene glycol 10 ml. The solution thus prepared was mixed with oil-in-water base 130 g. The cream thus prepared had pH 1.8 and contained 12 % gluconolactone. Alternatively, gluconolactone 15 g was dissolved in water 23 ml and the solution thus obtained was mixed with oil-in-water lotion 62 g. The lotion thus prepared had pH 1.8 and contained 15 % gluconolactone.

### EXAMPLE 18

A typical aldobionic acid cream was prepared as follows. Lactobionic acid 50% solution 20 g was mixed with oil-in-water base 80 g. The cream thus prepared had pH 2.1 and contained 10% lactobionic acid.

### EXAMPLE 19

A typical O-acetyl-hydroxyacid formulation for lip plumping was prepared as follows. O-Acetyl-mandelic acid 4 g was dissolved in ethanol 10 ml and the solution thus obtained was mixed with an ointment 86 g prepared from white petrolatum 50 parts, mineral oil 30 parts, spermaceti 5 parts, white beeswax 5 parts and isopropyl myristate 10 parts by weight. The ointment thus formulated contained 4% O-acetyl-mandelic acid.

### EXAMPLE 20

A female subject, age 22, applied topically twice daily gluconolactone 15% lotion to her right breast for 3 months. After 6 weeks, there was no change in the size of her untreated left breast, but she felt greater tightness of the right brassiere cup and found noticeable increase in the size of her right breast. At the end of 3 months, her right breast had increased in plumpness and firmness as compared to that of her left breast. At this time, she began treating both breasts. At the end of another 3 months, both breasts had substantially increased in plumpness and firmness. At the end of the 6 month treatment period, we concluded that the hydroxycarboxylic acid of the present invention is therapeutically effective for topical application to enlarge human breasts.

### EXAMPLE 21

Glyceric acid 14% cream was prepared as follows. L-Arginine 2.5 g was dissolved in 35 g D,L-glyceric acid 40% in water, and the solution thus obtained was mixed with 62.5 g hydrophilic ointment. The cream thus obtained had pH 3.5 and contained 14% glyceric acid. A female subject, age 58, presented with peri-oral skin atrophy, thin and drooping upper eyelids skin, and thin crepe paper-like skin of the upper arms. She was given the above glyceric acid 14% cream to be topically applied twice daily. After two months of such treatment, plumping of skin was detectable by increased skin thickness and appearance of fullness. The upper eyelidss had diminished droop and enhanced fullness. The lips had become more plump, and the skin of the upper arms had diminished crepe paper appearance.

Maltobionic acid 20% solution was prepared by dissolving maltobionic acid 20 g in ethanol 50 ml and propylene glycol 30 ml. The same subject topically applied twice daily the maltobionic acid solution prior to continued application of glyceric acid 14% cream. After five weeks, marked enhancement of skin fullness and plumping had occurred. The above results showed that PHAs used alone or in combination with ABAs are therapeutically beneficial for topical administration to plump, firm, enlarge or elongate mucous membrane and skin organs and sites which include lips, eyelids, breasts and penis.

### EXAMPLE 22

A combination lotion containing two PHAs was prepared as follows. D-Gluconolactone 3 g was dissolved in 17.5 g D,L-glyceric acid 40% in water, and the solution thus obtained was mixed with an oil-in-water lotion 79.5 g. A female subject, age 44, with thin upper eyelids which had a crepe paper like appearance was given the above combination lotion to be topically applied to the eyelids twice daily. After four months of use, the upper eyelids had lost the crepe paper appearance, and were smooth and more filled out in appearance. Before treatment, photographs showed a distinct concave appearance of the eyelids beneath the eyebrow. After four months of treatment, the skin of the upper eyelids had completely reversed the under-brow concave appearance.

The above results reveal that PHAs in combination with one another are therapeutically beneficial for topical administration to enlarge, plump or elongate mucous membrane and skin organs and sites which include lips. Vulva, penis, eyelids and breasts.

### EXAMPLE 23

A combination composition comprising an active ingredient of the embodiments described herein and a cosmetic agent for synergetic or synergistic effects was formulated as follows. D-Gluconolactone 15 g was dissolved in water 20 ml and 1 M L-arginine 10 ml, and the solution thus obtained was mixed with an oil-in-water emulsion or hydrophilic ointment 53 g. Vitamin E acetate 2 g was added and mixed with the above emulsion. The emulsion or cream thus prepared had pH 2.6 and contained 15% D-gluconolactone and 2% vitamin E acetate in an amphoteric composition. Both gluconolactone and vitamin E acetate are antioxidant substances and they provided synergetic or synergistic effects when the composition was topically applied to plump, firm, enlarge or elongate lips, eyelids, breasts or penis.

### EXAMPLE 24

A combination composition comprising an active ingredient of the embodiments described herein and a pharmaceutical agent for synergetic or synergistic effects was formulated as follows. Lactobionic acid 50% in water solution, 20 g and concentrated ammonium hydroxide 0.5 ml were mixed with an oil-in-water emulsion or hydrophilic ointment 59.3 g. Estrone or estradiol 0.2 g was dissolved in triethyl citrate 20 g, and the solution thus obtained was mixed with the above emulsion. The emulsion or cream thus prepared had pH 3.1 and contained 10% lactobionic acid, 0.2% estrone or estradiol and 20% triethyl citrate. Both estrone and estradiol are estrogenic hormones, and can provide synergetic or synergistic effects to plump, firm or enlarge lips, eyelids or breasts.

### EXAMPLE 25

A combination composition comprising an active ingredient of the embodiments described herein and a pharmaceutical agent for synergetic or synergistic effects was formulated as follows. N-Acetyl-α-D-glucosamine 10 g was dissolved in water 20 ml, and the solution thus obtained was mixed with an oil-in-water emulsion or hydrophilic ointment 59.5 g. Testosterone 0.5 g was dissolved in ethanol 10 ml, and the solution thus obtained was mixed with the above emulsion. The emulsion or cream thus prepared had pH 3.6 and contained 10% N-acetyl-glucosamine and 0.5% testosterone. Testosterone is an androgenic hormone and can provide synergetic or synergistic effects with an active ingredient of the present invention to plump, firm or elongate lips, eyelids or penis.

### EXAMPLE 26

One test on direct augmentation of penis was carried out as follows. Lactobionic acid 20% solution was prepared by dissolving lactobionic acid 20 g in water 50 ml and propylene glycol 30 ml. A male subject, age 81, volunteered to test an active ingredient of the present invention to enhance penile enlargement. Each morning for one week prior to treatment penile circumference was measured. Measurements were made with a cloth tailor's tape measure. Penile circumference of the glans region, which included the covering foreskin, ranged from 9.5 cm to 10.5 cm. The above lactobionic acid 20% solution was topically applied two to three times daily on the glans penis including the covering foreskin region for six weeks. Morning measurements were again made daily for one week after treatment was stopped. Penile circumference ranged from 11.5 cm to 13 cm after six weeks of topical treatment with 20% lactobionic acid solution. The results of this experiment reveal that glans penile augmentation had occurred, as well as increased thickness of foreskin.

The examples illustrated above reveal that hydroxycarboxylic acids, N-acyl-aldosamines, N-acylamino acids and related compounds on topical application to the affected area are useful to plump and pout lips, enhance and firm eyelids, enlarge and augment breasts, and elongate and expand the penis.

While the invention has been described with reference to particularly preferred embodiments, those skilled in the art will appreciate that various modifications may be made to the invention without significantly departing from the spirit and scope thereof.

1. A method of enlarging mucocutaneous or cutaneous organs and sites comprising topically applying a composition for an effective period of time to the mucocutaneous or cutaneous organ or site, the composition comprising an effective amount of at least one compound selected from the group consisting of hydroxycarboxylic acids, N-acyl-aldosamines, N-acylamino acids and related compounds.

2. The method as claimed in claim 1, wherein the hydroxycarboxylic acid is selected from the group consisting of alpha-hydroxyacids, beta-hydroxyacids, polyhydroxy acids, aldobionic acids and mixtures thereof, wherein the hydroxycarboxylic acid is present as a free acid, a salt, an amide, an ester or a lactone threof.

3. The method as claimed in claim 2, wherein the alpha-hydroxyacid is selected from the group consisting of alkyl alpha hydroxyacids, aralkyl alphahydroxyacids, polycarboxy alpha hydroxyacids, and mixtures thereof.

4. The method as claimed in claim 3, wherein the alkyl alpha hydroxyacid is represented by the formula:
R₁R₂C(OH)COOH
where R₁ and R₂ may be independently H or alkyl group, and wherein the alkyl AHA can exist as free acid, salt or partial salt with organic or inorganic alkali, amide, ester, lactone, stereoisomers as D, L and DL or R, S and RS forms when R₁ and R₂ are not identical, and wherein the alkyl groups are non-aromatic radicals selected from the group consisting of methyl, ethyl, propyl, isopropyl, butyl, pentyl, octyl, lauryl and stearyl.

5. The method as claimed in claim 4, wherein the alkyl AHA is selected from the group consisting of 2-hydroxyethanoic acid (glycolic acid); 2-hydroxypropanoic acid (lactic acid); 2-methyl-2-hydroxypropanoic acid (methyllactic acid); 2-hydroxybutanoic acid; 2-hydroxypentanoic acid; 2-hydroxyhexanoic acid; 2-hydroxyheptanoic acid; 2-hydroxyoctanoic acid; 2-hydroxyeicosanoic acid (alpha hydroxyarachidonic acid); 2-hydroxytetraeicosanoic acid (cerebronic acid); and 2-hydroxytetraeicosenoic acid (alpha hydroxynervonic acid).

6. The method as claimed in claim 3, wherein the aralkyl alphahydroxyacid is represented by the formula:
R₁R₂C(OH)COOH
where R₁ and R₂ may be independently H, aryl or aralkyl group, and wherein the aralkyl AHAs are present as a free acid, salt or partial salt with organic or inorganic alkali, amide, ester, lactone, stereoisomers as D, L and DL or R, S and RS forms when R₁ and R₂ are not identical, and wherein the hydroxyl group is attached to a non-aromatic alpha carbon atom.

7. The method as claimed in claim 6, wherein the aralkyl alpha hydroxyacid is selected from the group consisting of 2-phenyl-2-hydroxyethanoic acid (mandelic acid); 2,2-diphenyl-2-hydroxyethanoic acid (benzilic acid); 3-phenyl 2-hydroxypropanoic acid (3-phenyllactic acid); and 2-phenyl-2-methyl-2-hydroxyethanoic acid (atrolactic acid, 2-phenyllactic acid).

8. The method as claimed in claim 3, wherein the polycarboxy alpha hydroxyacids is represented by the formula:
R₁R₂C(OH)COOH
where R₁ and R₂ may be independently H, COOH, CH₂COOH or CHOHCOOH, and where the polycarboxy AHA are present as a free acid, salt or partial salt with organic or inorganic alkali, amide, ester, lactone, stereoisomers as D, L and DL or R, S and RS forms when R₁ and R₂ are not identical.

9. The method as claimed in claim 8, wherein the polycarboxy AHA is selected from the group consisting of 2-hydroxypropane-1,3-dioic acid (tartronic acid); 2-hydroxybutane-1,4-dioic acid (malic acid); 2,3-dihydroxybutane-1,4-dioic acid (tartaric acid); 2-hydroxy-2-carboxypentane -1,5-dioic acid (citric acid); and isocitric acid.

10. The method as claimed in claim 2, wherein the betahydroxyacid is represented by the formula:
R₁R₂C(OH)CHR₃COOH
where R₁, R₂, R₃ may be H, alkyl, aryl or aralkyl group, and wherein the beta hydroxyacid is present as a free acid, salt or partial salt with organic or inorganic alkali, amide, ester, lactone, as a stereoisomer as D, L and DL or R, S and RS forms when R₁ and R₂ are not identical or R₃ is not H.

11. The method as claimed in claim 10, wherein the betahydroxyacid is selected from the group consisting of: 3-hydroxypropanoic acid (,β-hydroxypropanoic acid); 3-hydroxybutanoic acid (β-hydroxybutanoic acid); 3-hydroxypentanoic acid; and 3-hydroxy-2-phenylpropanoic acid (tropic acid).

12. The method as claimed in claim 2, wherein the polyhydroxy acid PHAs is an organic carboxylic acids having multiple hydroxyl groups in addition to the alpha-hydroxyl group, and wherein the polyhydroxy acid is present in the lactone form.

13. The method as claimed in claim 12, wherein the polyhydroxy acid is derived from carbohydrates and is selected from the group consisting of aldonic acid, aldaric acid and alduronic acid.

14. The method as claimed in claim 13, wherein the aldonic acid is represented by the formula:
R(CHOH)ₙCHOHCOOH
where R is usually H or alkyl group; n an integer from 1-6, wherein the aldonic acid is present as a free acid, salt or partial salt with organic or inorganic alkali, amide, ester, lactone, and as a stereoisomer as D, L and DL or R, S and RS forms.

15. The method as claimed in claim 14, wherein the aldonic acid is selected from the group consisting of: 2,3-dihydroxypropanoic acid (glyceric acid); 2,3,4-trihydroxybutanoic acids (stereoisomers; erythronic acid and erythronolactone, threonic acid and threonolactone); 2,3,4,5-tetrahydroxypentanoic acids (stereoisomers; ribonic acid and ribonolactone, arabinoic acid and arabinolactone, xylonic acid and xylonolactone, lyxonic acid and lyxonolactone); 2,3,4,5,6-pentahydroxyhexanoic acids (stereoisomers; allonic acid and allonolactone, altronic acid and altronolactone, gluconic acid and gluconolactone, mannoic acid and mannolactone, gulonic acid and gulonolactone, idonic acid and idonolactone, galactonic acid and galactonolactone, talonic acid and talonolactone); and 2,3,4,5,6,7-hexahydroxyheptanoic acids (stereoisomers; alloheptonic acid and alloheptonolactone, altroheptonic acid and altroheptonolactone, glucoheptonic acid and glucoheptonolactone, mannoheptonic acid and mannoheptonolactone, guloheptonic acid and guloheptonolactone, idoheptonic acid and idoheptonolactone, galactoheptonic acid and galactoheptonolactone, taloheptonic acid and taloheptonolactone).

16. The method as claimed in claim 13, wherein the aldaric acid is represented by the formula:
HOOC(CHOH)ₙCHOHCOOH
where n is an integer from 1-4, and wherein the aldaric acid is present as a free acid, salt or partial salt with organic or inorganic alkali, amide, ester, lactone, or as a stereoisomer as D, L and DL or R, S and RS forms.

17. The method as claimed in claim 16, wherein the aldaric acid is selected from the group consisting of: 2,3-dihydroxybutane-1,4-dioic acids (stereoisomers; erythraric acid and threaric acid, also known as tartaric acid); 2,3,4-trihydroxypentane-1,5-dioic acids (stereoisomers; ribaric acid and ribarolactone, arabaric acid and arabarolactone, xylaric acid and xylarolactone, lyxaric acid and lyxarolactone); 2,3,4,5-tetrahydroxyhexane-1,6-dioic acids (stereoisomers; allaric acid and allarolactone, altraric acid and altrarolactone, glucaric acid and glucarolactone, mannaric acid and mannarolactone, gularic acid and gularolactone, idaric acid and idarolactone, galactaric acid and galactarolactone, talaric acid and talarolactone); 2,3,4,5,6-pentahyd roxyheptane- 1 ,7-dioic acids (stereoisomers; alloheptaric acid and alloheptarolactone, altroheptaric acid and altroheptarolactone, glucoheptaric acid and glucoheptarolactone, mannoheptaric acid and mannoheptarolactone, guloheptaric acid and guloheptarolactone, idoheptaric acid and idoheptarolactone, galactoheptaric acid and galactoheptarolactone, taloheptaric acid and taloheptarolactone).

18. The method as claimed in claim 13, wherein the alduronic acid is represented by the formula:
HOOC(CHOH)ₙCHOHCHO
where n is an integer from 1-4, wherein the alduronic acids is present as a free acid, salt or partial salt with organic or inorganic alkali, amide, ester, lactone, or as a stereoisomer as D, L and DL or R, S and RS forms.

19. The method as claimed in claim 18, wherein the alduronic acid is selected from the group consisting of: erythruronic acid and threuronic acid; riburonic acid and riburonolactone; araburonic acid and araburonolactone; xyluronic acid and xyluronolactone; lyxuronic acid and lyxuronolactone; alluronic acid and alluronolactone; altruronic acid and altruronolactone; glucuronic acid and glucuronolactone; mannuronic acid and mannuronolactone; guluronic acid and guluronolactone; iduronic acid and iduronolactone; galacturonic acid and galacturonolactone; taluronic acid and taluronolactone; allohepturonic acid and allohepturonolactone; altrohepturonic acid and altrohepturonolactone; glucohepturonic acid and glucohepturonolactone; mannohepturonic acid and mannohepturonolactone; gulohepturonic acid and gulohepturonolactone; idohepturonic acid and idohepturonolactone; galactohepturonic acid and galactohepturonolactone; and talohepturonic acid and talohepturonolactone.

20. The method as claimed in claim 3, wherein the aldobionic acid is represented by the following formula:
H(CHOH)ₘ(CHOR)(CHOH)ₙCOOH
where m and n are integers independently from 0-7, R is a monosaccharide, and wherein the aldobionic acid is present as a free acid, salt or partial salt with organic or inorganic alkali, amide, ester, lactone, or as a stereoisomer as D, L and DL or R, S and RS forms.

21. The method as claimed in claim 3, wherein the aldobionic acid is selected from the group consisting of: lactobionic acid and lactobionolactone from lactose, isolactobionic acid and isolactobionolactone from isolactose, maltobionic acid and maltobionolactone from maltose, isomaltobionic acid and isomaltobionolactone from isomaltose, cellobionic acid and cellobionolactone from cellobiose, gentiobionic acid and gentiobionolactone from gentiobiose, kojibionic acid and kojibionolactone from kojibiose, laminaribionic acid and laminaribionolactone from laminaribiose, melibionic acid and melibionolactone from melibiose, nigerobionic acid and nigerobionolactone from nigerose, rutinobionic acid and rutinobionolactone from rutinose, sophorobionic acid and sophorobionolactone from sophorose.

22. The method as claimed in claim 2, wherein the hydroxyacid is a hydroxyacid derivatives comprised of an ester form or an O-acetyl form of the hydroxyacid.

23. The method as claimed in claim 22, wherein the hydroxyacid derivative is selected from the group consisting of glycolic acid methyl ester and ethyl ester, O-acetyl-mandelic acid and O-acetyl-benzilic acid.

24. The method as claimed in claim 2, wherein the hydroxyacid is a related hydroxycarboxylic acid selected from alpha ketoacids and miscellaneous hydroxyacids.

25. The method as claimed in claim 24, wherein the alpha ketoacid is represented by the following formula:
(Ra)COCOOH
wherein Ra is H, alkyl, aralkyl or aryl group of saturated or unsaturated, isomeric or non-isomeric, straight or branched chain or cyclic form, having 1 to 25 carbon atoms, and in addition Ra may carry F, Cl, Br, I, OH, CHO, COOH and alkoxyl group having 1 to 9 carbon atoms.

26. The method as claimed in claim 25, wherein the alpha ketoacid is selected from the group consisting of: 2-ketoethanoic acid (glyoxylic acid), 2-ketopropanoic acid (pyruvic acid), 2-phenyl-2-ketoethanoic acid (benzoylformic acid), 3-phenyl-2-ketopropanoic acid (phenylpyruvic acid), 2-ketobutanoic acid, 2-ketopentanoic acid, 2-ketohexanoic acid, 2-ketoheptanoic acid, 2-ketooctanoic acid and 2-ketododecanoic acid.

27. The method as claimed in claim 24, wherein the miscellaneous hydroxyacid is selected from the group consisting of: agaricic acid, aleuritic acid, citramalic acid, glucosaminic acid, galactosaminic acid, 2-keto-gulonic acid and 2-keto-gulonolactone, mannosaminic acid, mevalonic acid and mevalonolactone, pantoic acid and pantolactone, quinic acid (1,3,4,5-tetrahydroxycyclohexanecarboxylic acid), piscidic acid (4-hydroxybenzyltartaric acid), isoascorbic acid (D-erythro-hex-2-enonic acidr-lactone), 2-hexulosonic acids (isomers; arabino-2-hexulosonicacid, xylo-2-hexulosonic acid, ribo-2-hexulosonic acid, lyxo-2-hexulosonic acid), 5-hexulosonic acids (isomers; arabino-5-hexulosonic acid, xylo-5-hexulosonic acid, ribo-5-hexulosonic acid, lyxo-5-hexulosonic acid), and mixtures thereof.

28. The method as claimed in claim 1, wherein the N-acyl-aldosamine is represented by the following formula:
R₁—(CHOH)ₘ—CH(NHCOR₂)—(CHOH)ₙ—R₃
where R₁ is selected from the group consisting of H, COOH, an alkyl, alkoxyl, aralkyl and aryl group having 1 to 19 carbon atoms, R₂ is selected from the group consisting of an alkyl, aralkyl or aryl group having 1 to 19 carbon atoms, m, n is independently an integer from 0-19, R₃ is selected from the group consisting of CHO, CONH₂, and COOR₄, R₄ is selected from the group consisting of H, an alkyl, aralkyl or aryl group having 1 to 9 carbon atoms, wherein the hydrogen attached to a carbon atom may be substituted by I, F, Cl, Br, or an alkyl, alkoxyl, aralkyl or aryl group having 1 to 19 carbon atoms, and wherein the N-Acyl-aldosamine is present as a saturated or unsaturated, stereoisomeric or non-stereoisomeric, straight or branched chain or cyclic form.

29. The method as claimed in claim 28, wherein the N-acyl-aldosamine is an N-acetyl-aldosamine selected from the group consisting of N-acetyl-glycerosamine, N-acetyl-erythrosamine, N-acetyl-threosamine, N-acetyl-ribosamine, N-Acetyl-arabinosamine, N-Acetyl-xylosamine, N-Acetyl-lyxosamine, N-Acetyl-allosamine, N-Acetyl-altrosamine, N-Acetyl-glucosamine, N-Acetyl-mannosamine, N-Acetyl-gulosamine, N-Acetyl-idosamine, N-Acetyl-galactosamine, N-Acetyl-talosamine, N-Acetyl-glucoheptosamine, N-Acetyl-galactoheptosamine, N-Acetyl-mannoheptosamine, N-acetyl-lactosamine, N-acetyl-muramic acid, N-acetyl-neuramine, N-acetyl-neuramin lactose, N-acetyl-glyceraminic acid, N-acetyl-erythrosaminic acid, N-acetyl-threosaminic acid, N-acetyl-ribosaminic acid, N-acetyl-arabinosaminic acid, N-acetyl-xylosaminic acid, N-acetyl-lyxosaminic acid, N-acetyl-allosaminic acid, N-acetyl-altrosaminic acid, N-acetyl-glucosaminic acid, N-acetyl-mannosaminic acid, N-acetyl-gulosaminic acid, N-acetyl-idosaminic acid, N-acetyl-galactosaminic acid, N-acetyl-talosaminic acid, N-acetyl-heptoglucosaminic acid, N-acetyl-heptogalactosaminic acid, N-acetyl-heptomannosaminic acid, N-acetyl-neuraminic acid (N-acetyl-nonulosaminic acid), N-acetyl-5-amino-5-deoxynonose), N-acetyl-hyalobiouronic acid, N-acetyl-chondrosine, N-acetyl-streptomycin, N-acetyl-erythromycin, N-acetyl-gentamycin, N-acetyl-nojirimycin, N-acetyl-glucosylamine, N-acetyl-mannosylamine, N-acetyl-galactosylamine, and mixtures thereof.

30. The method as claimed in claim 29, wherein the N-acetyl aldosamine is present as a five and six member ring form selected from the group consisting of 2-acetamido-2-deoxy-D-ribofuranoside, 2-acetamido-2-deoxy-D-ribopyranoside, 2-acetamido-2-deoxy-D-glucofuranoside, 2-acetamido-2-deoxy-D-glucopyranoside, 2-acetamido-2-deoxy -D-galactofuranoside and 2-acetamido-2-deoxy-D-galactopyranoside.

31. The method as claimed in claim 28, wherein the N-acyl-aldosamine is an N-Propanoyl-aldosamine selected from the group consisting of N-propanoyl-glycerosamine, N-propanoyl-erythrosamine, N-propanoyl-threosamine, N-propanoyl-ribosamine, N-Propanoyl-arabinosamine, N-Propanoyl-xylosamine, N-Propanoyl-lyxosamine, N-Propanoyl-allosamine, N-Propanoyl-altrosamine, N-Propanoyl-glucosamine (N-propanoyl-chitosamine), N-Propanoyl-mannosamine, N-Propanoyl-gulosamine, N-Propanoyl-idosamine, N-Propanoyl-galactosamine, N-Propanoyl-talosamine, N-Propanoyl-glucoheptosamine, N-Propanoyl-galactoheptosamine, N-Propanoyl-mannoheptosamine, N-propanoyl-lactosamine, N-propanoyl-muramic acid, N-propanoyl-neuramine, N-propanoyl-neuramin lactose, N-propanoyl-glyceraminic acid, N-propanoyl-erythrosaminic acid, N-propanoyl-threosaminic acid, N-propanoyl-ribosaminic acid, N-propanoyl-arabinosaminic acid, N-propanoyl-xylosaminic acid, N-propanoyl-lyxosaminic acid, N-propanoyl-allosaminic acid, N-propanoyl-altrosaminic acid, N-propanoyl-glucosaminic acid, N-propanoyl-mannosaminic acid, N-propanoyl-gulosaminic acid, N-propanoyl-idosaminic acid, N-propanoyl-galactosaminic acid, N-propanoyl-talosaminic acid, N-propanoyl-heptoglucosaminic acid, N-propanoyl-heptogalactosaminic acid, N-propanoyl-heptomannosaminic acid, N-propanoyl-neuraminic acid (N-propanoyl-nonulosaminic acid), N-propanoyl-5-amino-5-deoxynonose), N-propanoyl-hyalobiouronic acid, N-propanoyl-chondrosine, N-propanoyl-streptomycin, N-propanoyl-erythromycin, N-propanoyl-gentamycin, N-propanoyl-nojirimycin, N-propanoyl-glucosylamine, N-propanoyl-mannosylamine, N-propanoyl-galactosylamine, and other N-propanoyl-glycosylamines.

32. The method as claimed in claim 1, wherein the N-acylamino acid is represented by the following formula:
R₁—CH(NHCOR₂)—(CH₂)ₙ—COR₃
where R₁ is H, an alkyl, aralkyl or aryl group having 1 to 18 carbon atoms, R₂ is an alkyl, aralkyl or aryl group having 1 to 18 carbon atoms, n is an integer from 0 to 5, R₃ is OH, NH₂ or OR₃, and R₃ is an alkyl, aralkyl or aryl group having 1 to 9 carbon atoms, and in addition R₁ may carry OH, SH, SCH₃, COOH, NH₂, CONH₂, NHCONH₂, NHC(═NH)NH₂, imidazole, pyrrolidine or other heterocyclic group, and the hydrogen attached to a carbon atom may be substituted by I, F, Cl, Br, OH or alkoxyl group having 1 to 9 carbons.

33. The method as claimed in claim 32, wherein the N-acylamino acid is an N-acetylamino acid selected from the group consisting of N-acetyl-alanine, N-acetyl-arginine, N-acetyl-asparagine, N-acetyl-aspartic acid, N-acetyl-cysteine, N-acetyl-glycine, N-acetyl-glutamic acid, N-acetyl-glutamine, N-acetyl-histidine, N-acetyl-isoleucine, N-acetyl-leucine, N-acetyl-lysine, N-acetyl-methionine, N-acetyl-phenylalanine, N-acetyl-proline, N-acetyl-serine, N-acetyl-threonine, N-acetyl-tryptophan, N-acetyl-tyrosine and N-acetyl-valine.

34. The method as claimed in claim 1, wherein the related compounds are related N-Acetylamino acids selected from the group consisting of N-acetyl-β-alanine, N-acetyl-γ-aminobutanoic acid, N-acetyl-β-aminoisobutanoic acid, N-acetyl-citrulline, N-acetyl-dopa (N-acetyl-3,4-dihydroxyphenylalanine), N-acetyl-homocysteine, N-acetyl-homoserine, N-acetyl-ornithine, N-acetyl-phenylglycine, N-acetyl-4-hydroxyphenylglycine and N,O-diacetyl-4-hydroxyphenylglycine.

35. The method as claimed in claim 32, wherein the N-acylamino acid is an N-propanoyllamino acid selected from the group consisting of N-propanoyl-alanine, N-propanoyl-arginine, N-propanoyl-asparagine, N-propanoyl-aspartic acid, N-propanoyl-cysteine, N-propanoyl-glycine, N-propanoyl-glutamic acid, N-propanoyl-glutamine, N-propanoyl-histidine, N-propanoyl-isoleucine, N-propanoyl-leucine, N-propanoyl-lysine, N-propanoyl-methionine, N-propanoyl-phenylalanine, N-propanoyl-proline, N-propanoyl-serine, N-propanoyl-threonine, N-propanoyl-tryptophan, N-propanoyl-tyrosine and N-propanoyl-valine.

36. The method as claimed in claim 1, wherein the related compounds are related N-Propanoylamino Acids selected from the group consisting of N-propanoyl-β-alanine, N-propanoyl-γ-aminobutanoic acid, N-propanoyl-β-aminoisobutanoic acid, N-propanoyl-citrulline, N-propanoyl-dopa (N-propanoyl -3,4-dihydroxyphenylalanine), N-propanoyl-homocysteine, N-propanoyl -homoserine, N-propanoyl-ornithine, N-propanoyl-phenylglycine, N-propanoyl-4-hydroxyphenylglycine and N,O-dipropanoyl-4-hydroxyphenylglycine.

37. The method as claimed in claim 1, wherein the effective amount is from about 0.01% to about 99.9% by weight of the total composition.

38. The method as claimed in claim 37, wherein the effective amount is within the range of from about 0.1 to about 50% by weight.

39. The method as claimed in claim 37, wherein the effective amount is within the range of from about 1 to about 25% by weight.

40. The method as claimed in claim 1, wherein the effective period of time is for at least two weeks.

41. The method as claimed in claim 40, wherein the effective period of time is for at least two months.

42. The method as claimed in claim 40, wherein the effective period of time is for at least six months.

43. The method as claimed in claim 1, wherein the composition is topically applied to the lips to plump, pout, enhance, or enlarge the lips.

44. The method as claimed in claim 1, wherein the composition is applied to the eyelids to plump, firm, enhance, or enlarge the eyelids.

45. The method as claimed in claim 1, wherein the composition is applied to the breast to plump, enhance, or enlarge the breast.

46. The method as claimed in claim 1, wherein the composition is applied to the penis to plump, enhance, enlarge, and/or elongate the penis.

47. The method as claimed in claim 1, wherein the composition further comprises a cosmetic, pharmaceutical or other topical agent.

48. The method as claimed in claim 47, wherein the cosmetic, pharmaceutical or other topical agent is selected from the group consisting of agents that improve or eradicate age spots, keratoses and wrinkles; local analgesics and anesthetics; antiacne agents; antibacterials; antiyeast agents; antifungal agents; antiviral agents; antidermatitis agents; antihistamine agents; antipruritic agents; antiinflammatory agents; antipsoriatic agents; antiseborrheic agents; antiaging and antiwrinkle agents; sunblock and sunscreen agents; skin lightening agents; depigmenting agents; vitamins; corticosteroids; tanning agents; humectants; estrogens; androgens; hormones and retinoids.

49. The method as claimed in claim 47, wherein the cosmetic, pharmaceutical, or other topical agent is selected from the group consisting of aclovate, acyclovir, acetylsalicylic acid, adapalene, albuterol, aluminum acetate, aluminum chloride, aluminum hydroxide, aluminum chlorohydroxide, amantadine, aminacrine, aminobenzoic acid (PABA), aminocaproic acid, aminosalicylic acid, amitriptyline, anthralin, ascorbic acid, ascoryl palimate, atropine, azelaic acid, bacitracin, bemegride, beclomethasone dipropionate, benzophenone, benzoyl peroxide, betamethasone dipropionate, betamethasone valerate, brompheniramine, bupivacaine, butoconazole, calcipotriene, camphor, capsaicin, carbamide peroxide, chitosan, chlorhexidine, chloroxylenol, chlorpheniramine, ciclopirox, clemastine, clindamycin, clioquinol, clobetasol propionate, clotrimazole, coal tar, cromolyn, crotamiton, cycloserine, dehydroepiandrosterone, desoximetasone, dexamethasone, diphenhydramine, doxypin, doxylamine, dyclonine, econazole, erythromycin, estradiol, estrone, ethinyl estradiol, fluocinonide, fluocinolone acetonide, 5-fluorouracil, griseofulvin, guaifenesin, haloprogin, hexylresorcinol, homosalate, hydrocortisone, hydrocortisone 21-acetate, hydrocortisone 17-valerate, hydrocortisone 17-butyrate, hydrogen peroxide, hydroquinone, hydroquinone monoether, hydroxyzine, ibuprofen, ichthammol, imiquimod, indomethacin, ketoconazole, ketoprofen, kojic acid, lidocaine, meclizine, meclocycline, menthol, mepivacaine, methyl nicotinate, metronidazole, miconazole, minocycline, minoxidil, monobenzone, mupirocin, naftifine, naproxen, neomycin, nystatin, octyl methoxycinnamate, octyl salicylate, oxybenzone, oxiconazole, oxymetazoline, padimate O, permethrin, pheniramine, phenol, phenylephrine, phenylpropanolamine, piperonyl butoxide, podophyllin, podofilox, povidone iodine, pramoxine, prilocaine, procaine, promethazine propionate, propranolol, pseudoephedrine, pyrethrin, pyrilamine, resorcinol, retinal, 13-cis retinoic acid, retinoic acid, retinol, retinyl acetate, retinyl palmitate, selenium sulfide, shale tar, sulconazole, sulfur, sulfadiazine, tazarotene, testosterone, terbinafine, terconazole, tetracaine, tetracycline, tetrahydrozoline, thymol, tioconazole, tolnaftate, triamcinolone diacetate, triamcinolone acetonide, triamcinolone hexacetonide, triclosan, triprolidine, undecylenic acid, urea, vitamin E acetate, wood tar and zinc pyrithione.

50. A method of preventing or ameliorating breast tumors comprising topically applying to the breast for an effective period of time a composition comprising an effective amount of an antioxidant selected from the group consisting of citric acid, isocitric acid, malic acid, tartaric acid, pantolactone, isoascorbic acid, polyhydroxy acids, aldobionic acids and N-acetyl-cysteine.
